(19) United States
(12) Patent Application Publication
(10) Pub. No.: US 2025/0177375 A1
(43) Pub. Date: Jun. 5, 2025

(54) AZAPHILONE COMPOUND AND USE THEREOF IN PREPARATION OF ANTI-TUMOR DRUGS

(71) Applicant: OCEAN UNIVERSITY OF CHINA, Qingdao (CN)

(72) Inventors: Dehai LI, Qingdao (CN); Jing LI, Qingdao (CN); Chunxiao SUN, Qingdao (CN); Yu WU, Qingdao (CN); Qianqun GU, Qingdao (CN); Xin QI, Qingdao (CN); Tianjiao ZHU, Qingdao (CN); Qian CHE, Qingdao (CN); Guojian ZHANG, Qingdao (CN)

(21) Appl. No.: 18/842,265

(22) PCT Filed: Dec. 7, 2022
(86) PCT No.: PCT/CN2022/137267

§ 371 Date: Aug. 28, 2024

(30) Foreign Application Priority Data
Feb. 28, 2022 (CN) 202210186481.4

(51) Int. Cl. A61K 31/472; A61K 31/352; A61K 31/366; A61K 31/4741; A61P 35/04; C07D 217/04

(52) U.S. Cl. CPC A61K 31/472; A61K 31/352; A61K 31/366; A61K 31/4741; A61P 35/04; C07D 217/04

(57) ABSTRACT

Provided is a use of an azaphilone compound of formula (I) in the preparation of drugs for treating or preventing diseases, including but not limited to tumors, autoimmune diseases, infectious diseases and aging, and a method for treating the diseases by using the compounds. The present invention further relates to a novel azaphilone compound as an NK activator.

### FIELD OF THE INVENTION

The present invention relates to the technical field of immunotherapy drugs, particularly to the use of azaphilone compounds in the preparation of a medicament for the treatment or prevention of diseases such as tumors, and the method of treating diseases with said compounds, further relates to novel azaphilone compounds as NK activators.

### BACKGROUND

NK cells, the full name of Natural Killer cells, namely CD3−CD56+ lymphocytes, are important immune cells in the body. NK cells are associated with antitumor, antiviral, and immune regulation activities and, in some cases, with the occurrence of hypersensitivity and autoimmune diseases. NK cells make up approximately 10% of peripheral blood lymphocytes in healthy individuals, which recognizes diseased cells through killer immunoglobulin-like receptors (KIR) and co-stimulatory receptor-dependent, major histocompatibility complex (MHC)-independent mechanisms, killing abnormal cells within minutes without prior immune sensitization, thus making them as the first line of defense in the immune system due to its unique function.

Tumors are among the most significant diseases threatening human life and health. Immunotherapy has made breakthrough progress in recent years, mainly the development of CD8+ T cell checkpoint inhibitors, achieving an objective response rate of over 50% in some cancer patients. However, only 20% of patients have a response rate to this therapy, and even those who do may develop acquired resistance, necessitating the need for new immunotherapies targeting different pathways and effector cells.

Activated NK cells kill tumor cells both directly and indirectly: they release perforin and granzyme to lyse target cells; induce apoptosis through the tumor necrosis factor-related apoptosis-inducing ligand (TRAIL) and its receptor on target cells; and secrete cytokines like interferon to regulate innate and adaptive immune cells, thereby indirectly eliminating tumor cells. NK cells can enhance immune function in the body by complementing T cells in the immune system, due to their MHC independence.

NK cells are most in an immunosuppressive state within tumor tissues, and their quantity and activation levels significantly correlate with overall survival, progression-free survival, and relapse among patients. Thus, re-increasing and activating NK cells holds significant potential for tumor immunotherapy. NK cell activation relies on various expensive cytokines, which often cause widespread toxicity in vivo. Currently, there is a lack of effective small-molecule NK activators.

### SUMMARY

In prior research (Sun C.; Ge X.; Mudassir S.; Zhou L.; Yu G.; Che Q.; Zhang G.; Peng J.; Gu Q.; Zhu T. New glutamine-containing azaphilone alkaloids from deep-sea-derived fungus Chaetomium globosum HDN151398. Mar. Drugs 2019, 17, 253.), the inventors discovered compound 1778. However, disappointingly, subsequent pharmacological studies showed that the compound had an IC₅₀>50 pM in all in vitro assays of cancer cell lines. In the pharmaceutical field, this compound was considered to be inactive (see, for example, Preparation and Submission of Manuscripts, (Revised December 2012), Journal of Natural Products) and had no prospects for development and research as a drug.

However, further studies unexpectedly revealed that compound 1778 and other azaphilone analogs could efficiently (e.g., at concentrations as low as 1-100 nM) activate NK cells, which could be used to treat diseases treatable by NK cell activation, including but not limited to autoimmune diseases, tumors such as cancer and other diseases.

Therefore, in one aspect, this invention provides a compound of formula I or its pharmaceutically acceptable salts, stereoisomers

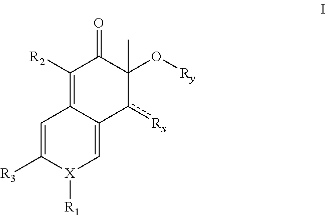

wherein
R₁ can be absent, HOC₁-C₆-alkyl-, —C₁-C₆-alkyl-COOR₇, wherein the C₁-C₆-alkyl is optionally substituted by one, two, or three groups independently selected from: —OH, C₁-C₆-alkyl-, C₁-C₆-alkyl- optionally substituted with —OH, C₆-C₁₀-aryl- optionally substituted with —OH, C₆-C₁₀-aryl-C₁-C₆-alkyl- optionally substituted with —OH, —C₁-C₆-alkyl-COOR₈, 5-10 membered heteroaryl- optionally substituted with —OH and 5-10 membered heteroaryl-C₁-C₆-alkyl- optionally substituted with —OH;
X can be O or N; provided that when X is O, R₁ is absent;
R₂ is halogen;
R₃ is selected from C₁-C₆ alkyl, C₂-C₆ alkenyl, which is optionally substituted by one, two, or three groups selected from C₁-C₃ alkyl and —OH;
[symbol] indicates a single or double bond;
Rₓ is =O and R_{y} is R₄;
or Rₓ and R_{y} together represent a structure:

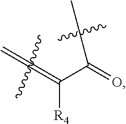

wherein the carbonyl carbon is connected to the O atom shown in formula I; or

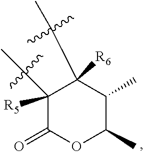

wherein the carbon carrying R₆ is connected to the O atom shown in formula I;
R₄ is selected from —CO—C₁-C₆ alkyl and —CO—C₂-C₆ alkenyl;
R₅ and R₆ are independently H or OH;
or R₅ and R₆ together form a double bond between the two carbon atoms to which they are attached;
R₇ and R₈ are independently selected from H and C₁-C₆-alkyl.

Use in the preparation of a medicament for treating diseases treatable by NK cell activation.

In one embodiment, R₁ is absent, HOC₁-C₃-alkyl-, —C₁-C₃-alkyl-COOR₇, wherein the C₁-C₃-alkyl is optionally substituted by one group independently selected from: —OH, C₁-C₃-alkyl-, C₁-C₃-alkyl- optionally substituted with —OH, phenyl optionally substituted with —OH, phenyl-C₁-C₃-alkyl- optionally substituted with —OH, —C₁-C₃-alkyl-COOR₈, 5-10 membered heteroaryl containing one or two heteroatoms selected from N, O, or S optionally substituted with —OH, and 5-10 membered heteroaryl-C₁-C₃-alkyl- containing one or two heteroatoms selected from N, O, or S optionally substituted with —OH; preferably, the 5-10 membered heteroaryl is selected from: indolyl, isoindolyl, pyrazolyl, imidazolyl, pyridyl, and oxazolyl;
R₃ is selected from C₄-C₆ alkyl, C₄-C₆ alkenyl, which is optionally substituted by one, two, or three C₁-C₃ alkyl groups or one —OH group;
R₇ and R₈ are each independently selected from H and C₁-C₃ alkyl;

The remaining variables are defined as above.

In one embodiment, R₁ is absent, —CH₂CH₂OH, —CH₂OOH,

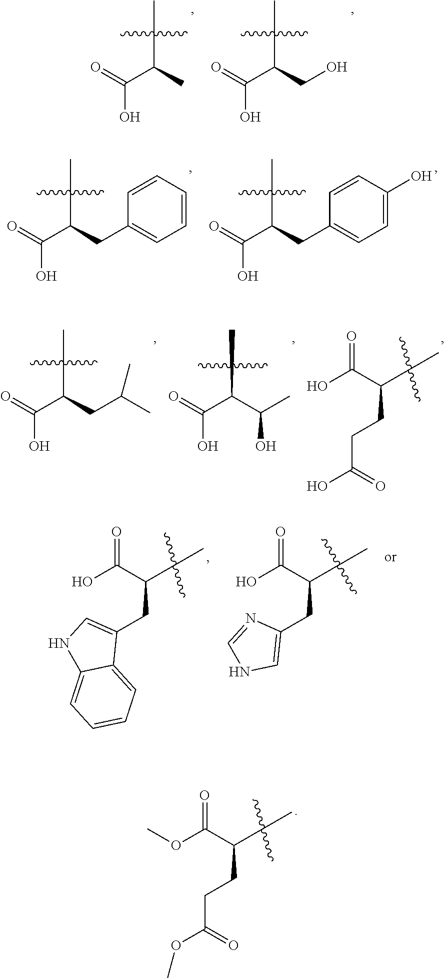

X is O or N; provided that when X is O, R₁ is absent;
R₂ is Cl;
R₃ is selected from

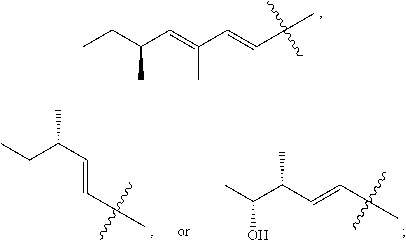

R₄ is CH₃CO— or

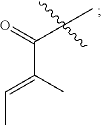 [SMILES: *C(=O)/C(C)=C/C]

R₅ is H;
R₆ is OH;
or R₅ and R₆ are connected together to form a double bond between the two carbon atoms to which they are attached.

In one embodiment, the compound of formula I has the structure of formula I′:

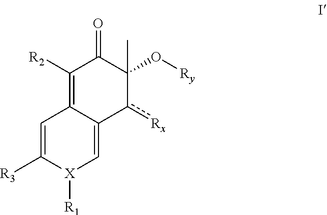

Wherein each variable (e.g., R₁, R₂, R₃, Rₓ, R_{y}, X) is as defined above.

In one embodiment, the compound of formula I has the structure of formula I-I, I-II, or I-III:

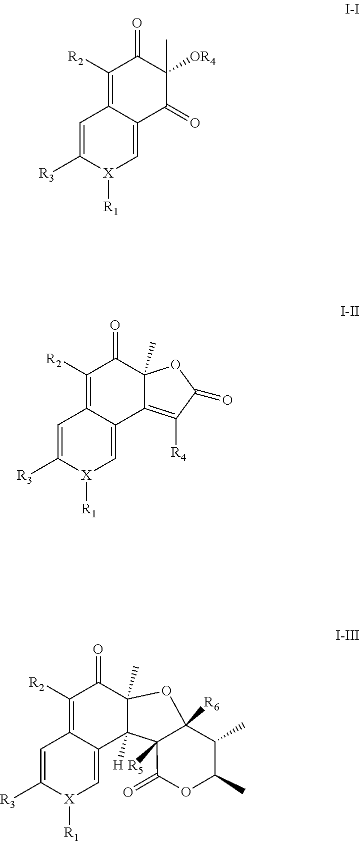

wherein each variable (e.g., R₁, R₂, R₃, R₄, R₅, R₆, X) is as defined above.

In one embodiment, the compound of formula I has the structure of formula I-I, I-II, or I-III:

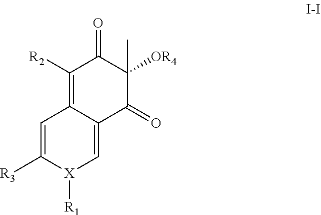

wherein
R₁ is absent, HOC₁-C₃ alkyl, or —C₁-C₃ alkyl-COOH, wherein the C₁-C₃ alkyl is optionally substituted by one group independently selected from: —OH, C₁-C₃-alkyl-, C₁-C₃-alkyl-optionally substituted with —OH, phenyl optionally substituted with —OH, and phenyl-C₁-C₃-alkyl- optionally substituted with —OH;
X is O or N, provided that when X is O, R₁ is absent;
R₂ is halogen;
R₃ is C₄-C₆ alkenyl, which is optionally substituted by one, two, or three C₁-C₃ alkyl groups, preferably

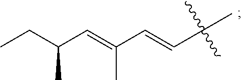 [SMILES: */C=C/C(C)=C/[C@@H](C)CC]

R₄ is —CO—C₁-C₆ alkyl, preferably CH₃CO—;
or

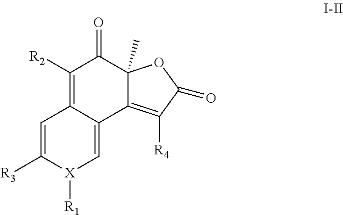

R₁ is —C₁-C₃ alkyl-COOR₇, wherein the C₁-C₃-alkyl is optionally substituted by one group independently selected from: —OH, —C₁-C₃-alkyl-COOR₈, and 5-10 membered heteroaryl-C₁-C₃-alkyl- containing one or two heteroatoms selected from N, O, or S optionally substituted with —OH; preferably, the 5-10 membered heteroaryl is selected from: indolyl, isoindolyl, pyrazolyl, imidazolyl, pyridyl, and oxazolyl;
R₇ and R₈ are each independently selected from H and C₁-C₃-alkyl-;
X is N;
R₂ is halogen;
R₃ is C₄-C₆ alkenyl, which is optionally substituted by one, two, or three C₁-C₃ alkyl groups preferably

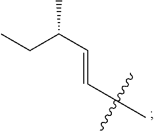 [SMILES: */C=C/[C@@H](C)CC]

R₄ is —CO—C₂-C₆ alkenyl, preferably

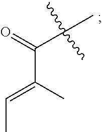 [SMILES: *C(=O)/C(C)=C/C]

or

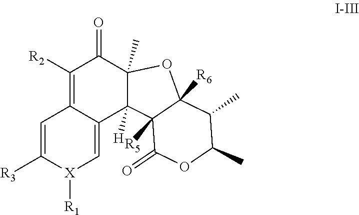

R₁ is absent;
X is O;
R₂ is halogen;
R₃ is C₄-C₆ alkenyl, optionally substituted by a group selected from hydroxy and C₁-C₃ alkyl, preferably

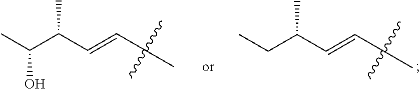

R₅ is H;
R₆ is OH;
or R₅ and R₆ are connected together to form a double bond between the two carbon atoms to which they are attached.

In one embodiment, the compound of formula I is selected from the following compounds:

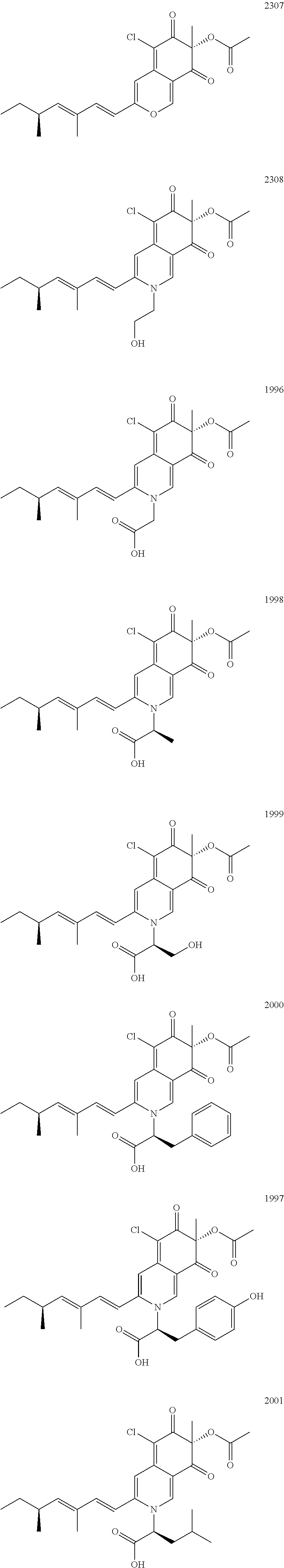

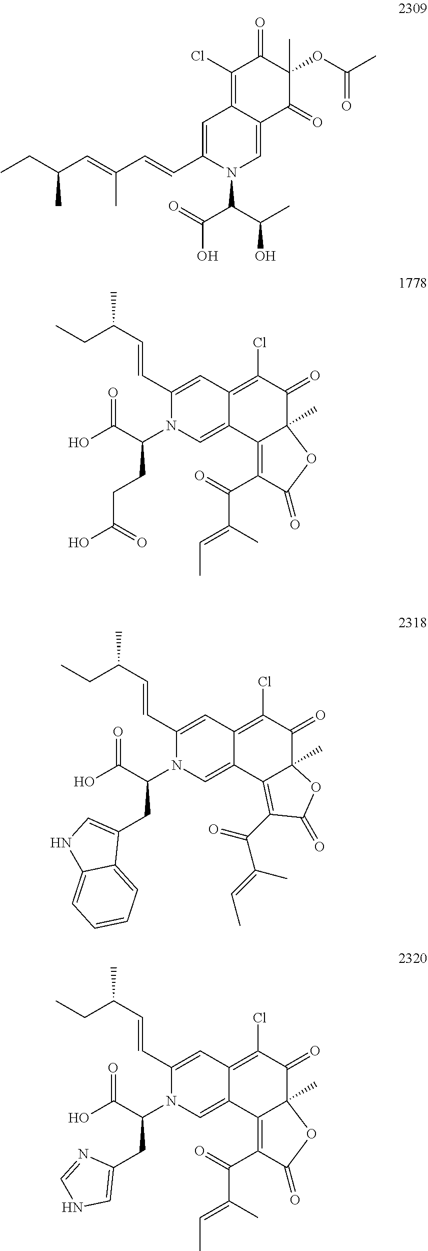

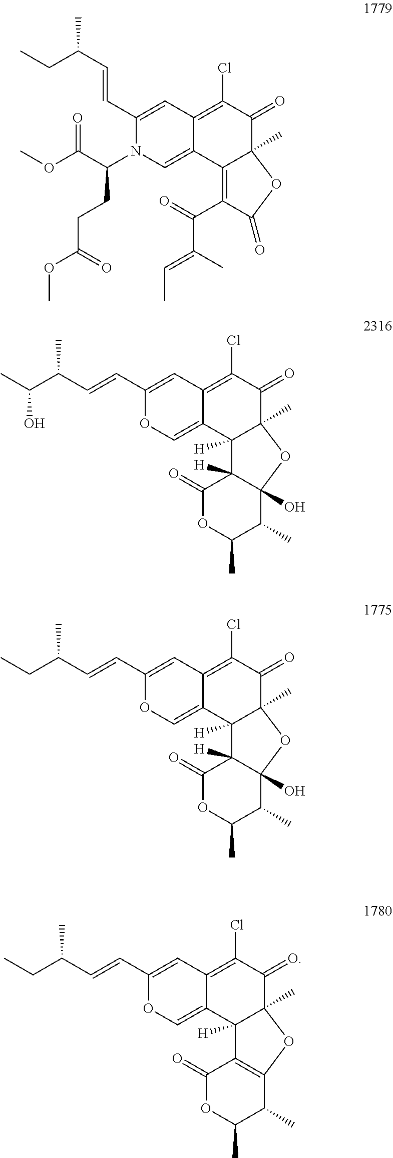

In one embodiment, the compound of formula I is

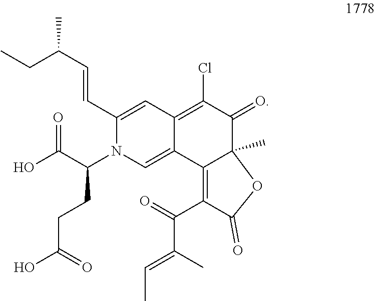 [SMILES: C/C=C(\C)C(=O)C1=C2C3=CN([C@@H](CCC(=O)O)C(=O)O)C(/C=C/[C@@H](C)CC)=CC3=C(Cl)C(=O)[C@@]2(C)OC1=O]

In one embodiment, diseases treatable by NK cell activation include, but are not limited to: autoimmune diseases, infectious diseases, tumors, myelofibrosis, acute/chronic graft-versus-host disease, and aging;
preferably including, but not limited to: tumors such as liver cancer, kidney cancer, gastric cancer, colon cancer, lung cancer, ovarian cancer, pancreatic cancer, prostate cancer, breast cancer, melanoma, thyroid cancer, glioma, multiple myeloma; leukemia such as myeloid leukemia, lymphoid leukemia, erythroid leukemia, lymphoma; autoimmune diseases such as rheumatoid arthritis, systemic lupus erythematosus, Sjögren's syndrome; viral infections such as HBV, HCV, HIV infections like hepatitis B, hepatitis C, acquired immunodeficiency syndrome (AIDS); bacterial infections such as enteritis, hepatitis, pneumonia, pancreatitis, prostatitis; and aging.

In one embodiment, the medicament is in the form of a unit dosage form with a pharmaceutically acceptable dose, with each unit dosage form containing 1-500 mg or 10-100 mg of the compound of formula I or its sub-formula compound or its pharmaceutically acceptable salts, stereoisomers.

In another aspect, the present invention provides the use of the compound of formula I or its sub-formula compound or its pharmaceutically acceptable salts, stereoisomers as described above for the preparation of a drug used as an NK activator, preferably for the treatment of diseases treatable by NK cell activation as described herein.

In another aspect, the present invention provides the use of the compound of formula I or its sub-formula compound or its pharmaceutically acceptable salts, stereoisomers as described above in the preparation of a medicament to enhance the killing ability of NK cell against tumor cells.

In another aspect, the present invention provides the compound of formula I or its sub-formula compound or its pharmaceutically acceptable salts, stereoisomers as described above for use as an NK activator.

In another aspect, the present invention provides the compound of formula I or its sub-formula compound or its pharmaceutically acceptable salts, stereoisomers as described above for the treatment of diseases treatable by NK cell activation as described herein.

In another aspect, the present invention provides the compound of formula I or its sub-formula compound or its pharmaceutically acceptable salts, stereoisomers as described above for promoting the proliferation of NK92, when administered at a concentration of 1-100 nM.

In another aspect, the present invention provides the compound of formula I or its sub-formula compound or its pharmaceutically acceptable salts, stereoisomers as described above for concentration-dependent enhancement of the killing ability of NK cell against tumor cells in NK92, normal human peripheral blood, and patient pleural effusion.

In another aspect, the present invention provides a method of treating a subject's disease, comprising administering to the subject a therapeutically effective amount of the compound of formula I or its sub-formula compound or its pharmaceutically acceptable salts, stereoisomers as described above.

In another aspect, the present invention provides a method of treating a subject's disease, comprising administering to the subject a therapeutically effective amount of the compound of formula I or its sub-formula compound or its pharmaceutically acceptable salts, stereoisomers as described above, wherein the disease is treatable by NK cell activation.

In one embodiment, the method further comprises administering NK cells to the subject, wherein the NK cells are isolated natural NK cells or artificially modified or prepared NK cells.

In another aspect, the present invention provides a combination product comprising the compound of formula I or its pharmaceutically acceptable salts, stereoisomers as described above, and NK cells, preferably the NK cells are isolated natural NK cells or artificially modified or prepared NK cells.

In one embodiment, the combination product comprises a pharmaceutically acceptable unit dosage of the compound of formula I or its sub-formula compound or its pharmaceutically acceptable salts, stereoisomers, with each unit dosage form containing 1-500 mg or 10-100 mg of the compound of formula I or its sub-formula compound or its pharmaceutically acceptable salts, stereoisomers.

In another aspect, the present invention provides the compound of formula I-I or its pharmaceutically acceptable salts, stereoisomers:

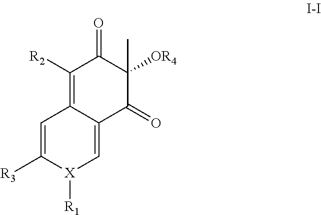

Wherein
R₁ is HOC₁-C₆-alkyl-, —C₁-C₆-alkyl-COOR₇, wherein the C₁-C₆-alkyl is optionally substituted by 1, 2, or 3 groups independently selected from: —OH, C₁-C₆-alkyl-, C₁-C₆-alkyl- optionally substituted with —OH, C₆-C₁₀-aryl- optionally substituted with —OH, C₆-C₁₀ aryl-C₁-C₆-alkyl- optionally substituted with —OH, 5-10 membered heteroaryl optionally substituted with —OH, and 5-10 membered heteroaryl-C₁-C₆-alkyl- optionally substituted with —OH;
X is N;
R₂ is halogen;
R₃ is selected from C₁-C₆ alkyl, C₂-C₆ alkenyl, which is optionally substituted by one, two, or three groups selected from C₁-C₃ alkyl and —OH;
R₄ is selected from —CO—C₁-C₆ alkyl and —CO—C₂-C₆ alkenyl;
R₇ is independently selected from H and C₁-C₆-alkyl.

In one embodiment, R₁ is HOC₁-C₃-alkyl- or —C₁-C₃-alkyl-COOR₇, wherein the C₁-C₃-alkyl is optionally substituted by one group independently selected from: —OH, C₁-C₃-alkyl-, C₁-C₃-alkyl- optionally substituted with —OH, phenyl- optionally substituted with —OH, phenyl-C₁-C₃-alkyl- optionally substituted with —OH, 5-10 membered heteroaryl containing one or two heteroatoms selected from N, O, or S optionally substituted with —OH, and 5-10 membered heteroaryl-C₁-C₃-alkyl- containing one or two heteroatoms selected from N, O, or S optionally substituted with —OH. Preferably, the 5-10 membered heteroaryl is selected from: indolyl, isoindolyl, pyrazolyl, imidazolyl, pyridyl, and oxazolyl.

R₃ is selected from C₄-C₆ alkyl and C₄-C₆ alkenyl, which is optionally substituted by one, two, or three C₁-C₃ alkyl or one —OH.

R₇ is independently selected from H and C₁-C₃-alkyl.

Other variables are defined as above.

In one embodiment, R₁ is HOC₁-C₃-alkyl- or —C₁-C₃ alkyl-COOH, wherein the C₁-C₃ alkyl is optionally substituted by one group independently selected from: —OH, C₁-C₃-alkyl-, C₁-C₃-alkyl- optionally substituted with —OH, phenyl- optionally substituted with —OH, and phenyl-C₁-C₃-alkyl- optionally substituted with —OH.

### X is N.

R₂ is a halogen.

R₃ is a C₄-C₆ alkenyl, which is optionally substituted by one, two, or three C₁-C₃ alkyl groups, preferably

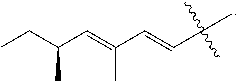 [SMILES: */C=C/C(C)=C/[C@@H](C)CC]

R₄ is —CO—C₁-C₆ alkyl, preferably CH₃CO—.

In one embodiment, R₁ is —CH₂CH₂OH, —CH₂OOH,

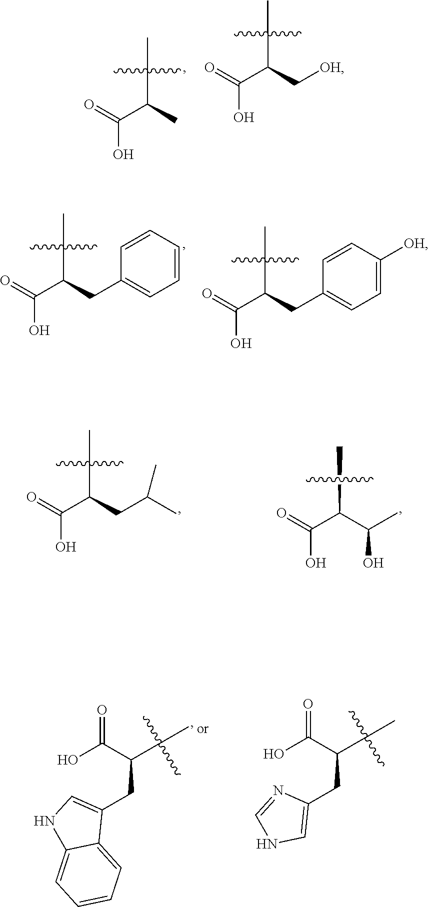

X is N;
R₂ is a halogen, such as Cl;
R₃ is selected from C₄-C₆ alkenyl, which is optionally substituted by one, two, or three C₁-C₃ alkyl groups, preferably

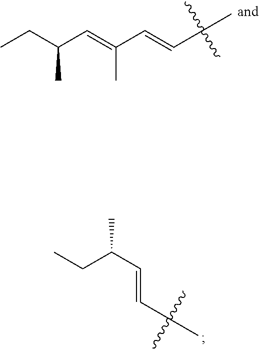

R₄ is —CO—C₁-C₃ alkyl or —CO—C₂-C₄ alkenyl.

In one embodiment, the compound of formula I is selected from the following compounds:

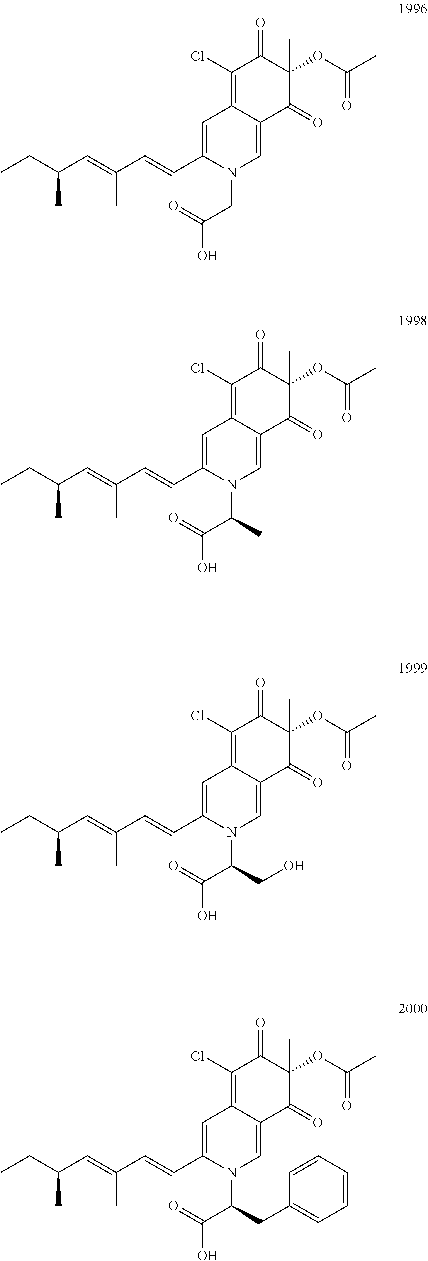

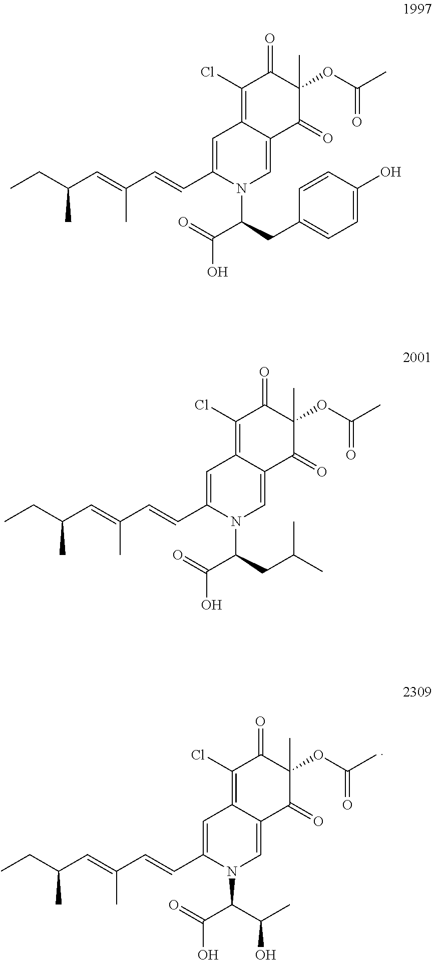

In another aspect, the invention provides a pharmaceutical composition comprising the compound of formula I as described above or its pharmaceutically acceptable salts, stereoisomers, and a pharmaceutically acceptable excipient.

In one embodiment, the pharmaceutical composition contains a pharmaceutically acceptable unit dose of the compound of formula I or its subformula compound or its pharmaceutically acceptable salts, stereoisomers, for example, with each unit dosage form containing 1-500 mg, 10-100 mg of the compound of formula I or its subformula compound or its pharmaceutically acceptable salts, stereoisomers.

In one embodiment, the pharmaceutical composition is in the form of any one of the following: suspensions, emulsions, tablets, capsules, granules, oral liquids, or injections.

### Definitions

The following terms, phrases, and/or symbols used in this application have the meanings ascribed to them below, unless otherwise specified in the context in which they are used.

A short hyphen (“-”) at the end of a group indicates the connection point of the group. For example, HOC₁-C₃-alkyl- or —C₁-C₃-alkyl-COOH means connecting through the alkyl to the rest of the molecule. It should be understood that, in many cases, the connection point of the substituent is known to those skilled in the art and the “-” can be omitted, such as for halogen substituents.

The term “alkyl” refers to a saturated hydrocarbon group containing 1-10 carbon atoms, preferably 1-6 carbon atoms, more preferably 1-4, 1-3, or 1-2 carbon atoms, which may be linear or branched. For example, “C₁₋₆ alkyl” means an alkyl group having 1-6 carbon atoms. Examples of alkyl groups include, but are not limited to, methyl, ethyl, propyl, isopropyl, n-butyl, isobutyl, tert-butyl, and n-pentyl.

It should be understood that an alkyl group with groups connected at both ends in the middle of a structure is actually an alkylene group. For example, in “—C₁-C₃-alkyl-COOR₇,” “—C₁-C₃-alkyl-” actually means “—C₁-C₃-alkylene-,” preferably “—CH₂—” or “—CH₂—CH₂-.” The representation “—C₁-C₃-alkyl-COOR₇” is recognized and accepted in the field. Other similar cases can be understood similarly.

The term “alkenyl” as used herein refers to an unsaturated hydrocarbon group containing one or more, e.g., 1, 2, or 3 carbon-carbon double bonds (C═C) and having 2-10 carbon atoms, preferably 2-6 carbon atoms, 2-5, 2-4, 3-5, 4-5, or 4-6 carbon atoms, which may be linear or branched. For example, “C₂₋₆ alkenyl” means an alkenyl group having one, two, or three preferably one or two carbon-carbon double bonds and 2-6 carbon atoms. Examples of alkenyl groups include, but are not limited to, vinyl, propenyl, allyl, 2-butenyl, isopentenyl, and pentadienyl. The connection point of the alkenyl group can be at the double bond or not.

The term “halogen” or “halo” as used herein refers to fluorine (—F), chlorine (—Cl), bromine (—Br), and iodine (—I), preferably fluorine, chlorine, and bromine, more preferably fluorine and chlorine.

The term “C₆-C₁₀ aryl” as used herein refers to a carbocyclic aromatic hydrocarbon group containing 6-10 carbon atoms, consisting of one or more (e.g., 2) fused rings. Examples of aryl groups include, but are not limited to, phenyl and naphthyl, preferably phenyl.

The term “5-10 membered heteroaryl” as used herein refers to an aromatic hydrocarbon group (i.e. 9-10 membered heteroaryl, 5-6 membered heteroaryl, 5-membered heteroaryl or 6-membered heteroaryl) having 5-10 ring atoms (e.g., 9-10 ring atoms, 5-6 ring atoms, 5 or 6 ring atoms), containing one or more (e.g., 1, 2, or 3, more preferably 1 or 2) ring heteroatoms independently selected from N, O, and/or S, with the remaining ring atoms being carbon atoms; it may have one or more rings, e.g., one, two, or three, preferably one or two rings. For example, the heteroaryl groups include, but are not limited to, indolyl, isoindolyl, pyrazolyl, imidazolyl, pyridyl, and/or oxazolyl.

The terms “option,” “optional,” or “optionally” as used herein mean that the subsequent described event or condition may or may not occur and the description includes cases where the event or condition occurs and where it does not occur. For example, “optionally substituted by” means that the involved group or atom is substituted or not substituted by the specified substituent. When it indicates “substituted by”, it means that the given atom or group is substituted by one, two, or three (preferably 1) substituents independently selected from the given groups (e.g., halogen and/or hydroxyl). It should be understood that the structures and/or substitutions involved herein presuppose compliance with valence rules and that such combinations are permissible only if combinations of substituents and/or variables result in chemically correct and stable compounds.

The term “pharmaceutically acceptable salt” as used herein refers to non-toxic, biologically tolerable or otherwise biologically suitable salts of the compound of formula (I) with free acids or bases, suitable for administration to an individual. These salts include acid addition salts and base addition salts. Acids commonly used to form acid addition salts include inorganic acids such as hydrochloric acid, hydrobromic acid, hydroiodic acid, sulfuric acid, phosphoric acid, etc., and organic acids such as tartaric acid, p-toluenesulfonic acid, methanesulfonic acid, oxalic acid, p-bromobenzenesulfonic acid, carbonic acid, succinic acid, citric acid, benzoic acid, acetic acid, oxalic acid, etc. Base addition salts include salts derived from inorganic bases such as ammonium or alkali metals or alkaline earth metals hydroxides, carbonates, bicarbonates, etc. Bases suitable for preparing salts of the invention include, but are not limited to, sodium hydroxide, potassium hydroxide, ammonium hydroxide, potassium carbonate, sodium carbonate, sodium bicarbonate, potassium bicarbonate, calcium hydroxide, calcium carbonate, etc. Organic salts of the invention include acetates, propionates, butyrates, tartrates, maleates, hydroxymaleates, fumarates, citrates, lactates, mucates, gluconates, benzoates, succinates, oxalates, phenylacetates, methanesulfonates, p-toluenesulfonates, benzenesulfonates, p-aminosalicylates, aspartates, glutamates, edetates, stearates, palmitates, oleates, laurates, pantothenates, tannates, ascorbates, valerates, or alkylammonium salts. For a general description of pharmaceutically acceptable salts, see for example: S. M. Berge et al., “Pharmaceutical Salts”, J. Pharm. Sci., 1977, 66:1-19, and Handbook of Pharmaceutical Salts, Properties, Selection, and Use, edited by Stahl and Wermuth, Wiley-VCH and VHCA, Zurich, 2002.

It should be understood by those skilled in the art that some compounds of formula (I) may contain one or more chiral centers and therefore exist as two or more stereoisomers. Racemic mixtures of these isomers, individual stereoisomers, and mixtures enriched in one enantiomer, as well as diastereomers and mixtures partially enriched by specific diastereomers when there are two chiral centers, are within the scope of the invention. It should also be understood that the invention includes all individual stereoisomers (e.g., enantiomers), racemic mixtures, or partially resolved mixtures of the compounds of formula (I), and, where appropriate, includes their individual tautomers.

The term “disease treatable by NK cell activation” as used herein includes, but is not limited to, tumors, autoimmune diseases, infectious diseases, and aging; and the diseases mentioned above;

Preferably, it includes, but is not limited to, tumors such as liver cancer, kidney cancer, gastric cancer, colon cancer, lung cancer, ovarian cancer, pancreatic cancer, prostate cancer, breast cancer, melanoma, thyroid cancer, glioma, multiple myeloma; leukemia such as myeloid leukemia, lymphoid leukemia, erythroid leukemia, lymphoma; autoimmune diseases such as rheumatoid arthritis, systemic lupus erythematosus, Sjögren's syndrome; viral infections such as HBV, HCV, HIV infections such as hepatitis B, hepatitis C, acquired immune deficiency syndrome (AIDS); bacterial infections: enteritis, hepatitis, pneumonia, pancreatitis, prostatitis; aging.

As defined herein, “NK cells” refers to natural killer cells, for example, in vivo (e.g., in humans) or in vitro, such as NK-92 cell line.

The term “pharmaceutically acceptable unit dose” as used herein refers to the amount of active ingredient contained in a unit dosage form suitable for administration to a subject, preferably a human, such as 1-1000 mg, 1-600 mg, 1-500 mg, 1-100 mg, 5-200 mg, 10-100 mg, 10-500 mg, 10-200 mg, 50-100 mg, for example, 1, 5, 10, 15, 20, 60, 80, 100, 150, 200, 250, 300, 400, 500, 600, 700 mg.

The term “therapeutically effective amount” as used herein refers to the amount of the compound of the invention that is typically sufficient to activate NK cells to produce a beneficial therapeutic effect in a patient suffering from a disease treatable by NK cell activation. This effective amount of the active ingredient of the invention can be determined by conventional methods (e.g., modeling, dose-escalation studies, or clinical trials) in conjunction with conventional influencing factors (e.g., route of administration, pharmacokinetics of the drug component, severity of the disease or disorder, prior or ongoing treatments of the individual, the individual's health condition and response to the drug, and the judgment of the attending physician).

Typical therapeutically effective amounts include doses such as approximately 0.0001 to about 100 mg of active ingredient per kilogram of body weight per day, for example, from about 0.001 to 100 mg/kg/day, 0.1 to 100 mg/kg/day, 1 to 50 mg/kg/day, 10 to 50 mg/kg/day, or about 1 to 5 mg/kg/day, or about 0.1 to 10 mg/kg, administered once daily or in divided doses (e.g., twice daily, three times daily, or four times daily). For a 70 kg person, suitable dose ranges include approximately 1 mg to about 1500 mg/day, or about 3 mg to about 500 mg/day, or about 5 mg to about 300 mg/day, or about 10 mg to about 200 mg/day, or about 1 mg to about 100 mg/day, or about 50 mg to about 200 mg/day.

The term “subject” as used herein refers to both mammals and non-mammals. Mammals refers to any member of the class of mammals, including but not limited to: humans; non-human primates such as chimpanzees and other apes and monkey species; farm animals such as cattle, horses, sheep, goats, and pigs; domestic animals such as rabbits, dogs, and cats; laboratory animals, including rodents such as rats, mice, and guinea pigs; and others. Examples of non-mammals include but are not limited to birds. The term “subject” is not limited to a specific age or sex. In some embodiments, the individual is human.

The term “pharmaceutically acceptable excipient” as used herein refers to one or more compatible solid or liquid fillers or gel substances that are pharmacologically inactive, compatible with other components of the composition, and acceptable for administration to warm-blooded animals such as humans, for example as carriers or vehicles for the compounds of the invention in a form for administration. Examples include but are not limited to cellulose and its derivatives (such as sodium carboxymethylcellulose, cellulose acetate, etc.), gelatin, talc, solid lubricants (such as magnesium stearate), calcium sulfate, vegetable oils, polyols (such as propylene glycol, glycerin, mannitol, sorbitol, etc.), emulsifiers (such as polysorbates), wetting agents (such as sodium dodecyl sulfate), colorants, flavoring agents, stabilizers, antioxidants, preservatives, etc.

As used herein, the term “combination product” refers to a fixed combination in a single unit dosage form or a kit of parts for combined administration, wherein the compound of formula I and the combination partner (e.g., NK cells) can be administered independently at the same time or separately at different time intervals, particularly when these intervals allow the combination partner to display collaboration such as synergy. The terms “co-administration” or “combined administration” or similar terms as used herein are intended to include the administration of the chosen combination partner to a single subject (e.g., patient) as needed, and are meant to encompass therapeutic regimens wherein the active agents do not have to be administered via the same route or at the same time. The “combination product” includes both fixed and non-fixed combinations of the compound of formula I and the combination partner (e.g., NK cells). The term “fixed combination” means that active ingredients such as the compound of formula I and the combination partner are administered to the patient as a single entity or dosage form simultaneously. The term “non-fixed combination” means that the active ingredients such as the compound of formula I and the combination partner are administered as separate entities either simultaneously, jointly, or sequentially without a specific time limit, wherein the administration provides a therapeutic effective level of both compounds in the patient. The latter also applies to cocktail therapies, such as administering three or more active ingredients. The “combination product” is preferably a “pharmaceutical combination product.”

### BENEFITS OF THE INVENTION

The azaphilone compounds of the present invention, particularly compound 1778, effectively (e.g., at concentrations as low as 1-100 nM) activate NK cells (e.g., NK92 cell lines, NK cells from normal human peripheral blood and tumor patient pleural effusions), enhance NK cell degranulation, release granzyme B, perforin, and interferon, improve NK cell-mediated killing function of tumor cells both in vivo and in vitro, and inhibit tumor growth and metastasis. It can be used to treat diseases treatable by NK cell activation, including but not limited to: tumors, autoimmune diseases, infectious diseases, and aging; the drug is effective with a broad anti-cancer spectrum.

### ILLUSTRATION DESCRIPTIONS

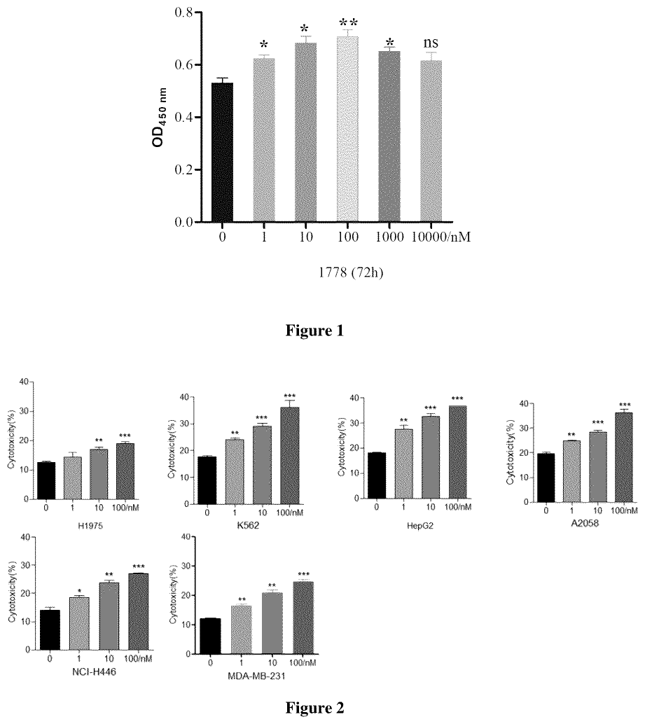

FIG. 1: Shows that compound 1778 promotes the proliferation of NK-92 cell line. *P<0.05, **P<0.01

FIG. 2: Shows that compound 1778 significantly promotes the killing activity of NK-92 cells. 1778 enhances the killing level of NK-92 cells against different types of tumor cells. *P<0.05, **P<0.01.

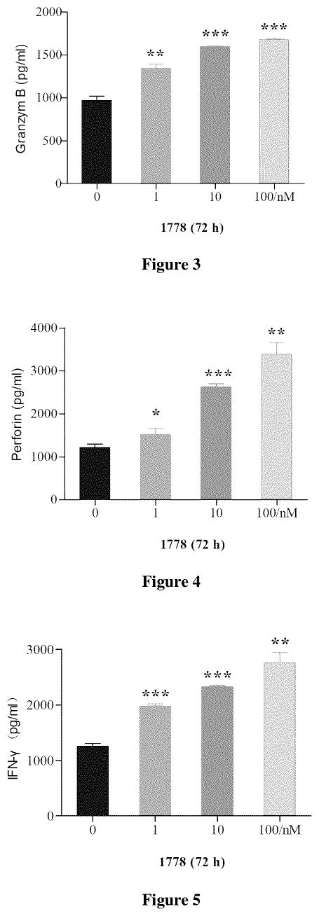

FIG. 3: Shows that compound 1778 significantly promotes the release of granzyme B by NK-92 cells. **P<0.01, ***P<0.001.

FIG. 4: Shows that compound 1778 significantly promotes the release of perforin by NK-92 cells. *P<0.05, **P<0.01, ***P<0.001.

FIG. 5: Shows that compound 1778 significantly promotes the release of IFN-γ by NK-92 cells. **P<0.01, ***P<0.001.

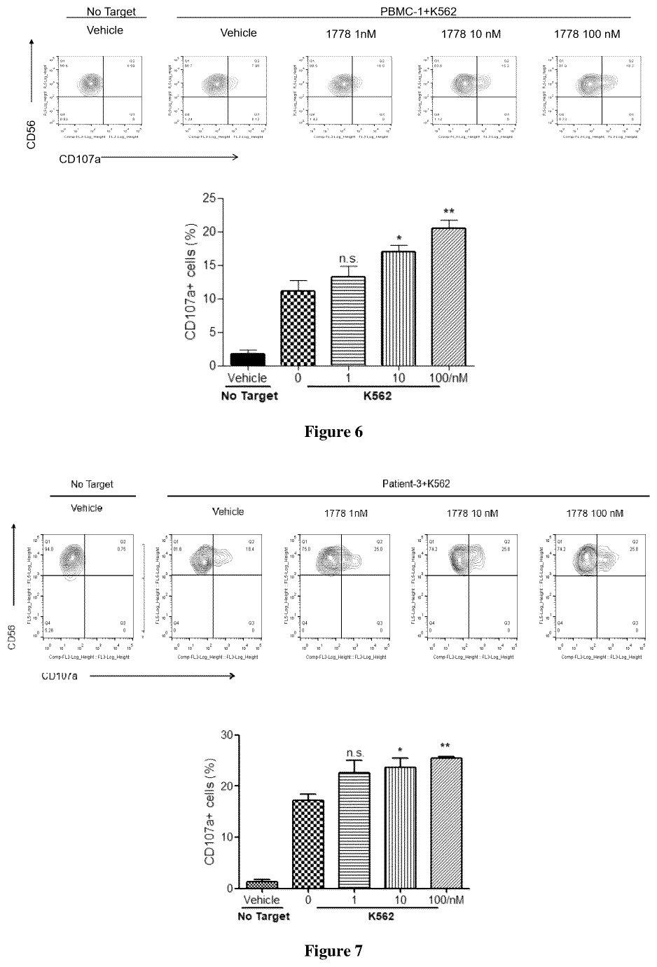

FIG. 6: Shows that compound 1778 significantly promotes the degranulation of PBMC cells in normal humans. *P<0.05, **P<0.01.

FIG. 7: Shows that compound 1778 significantly promotes the degranulation of PBMC cells extracted from the pleural effusion of lung cancer patients. *P<0.05, **P<0.01.

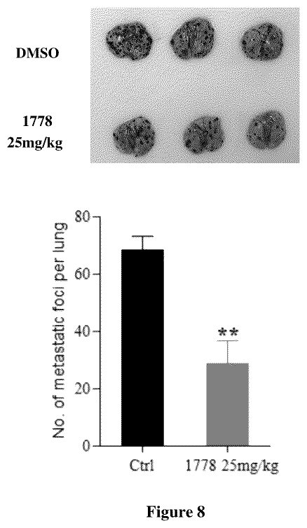

FIG. 8: Shows that compound 1778 has a significant inhibitory effect on the metastasis of melanoma cells in vivo. **P<0.01.

### DETAILED IMPLEMENTATION METHODS

The technical solutions of the invention is described clearly and completely in combination with specific examples of the invention, but the scope of protection of the present invention is not limited to these examples. Any modifications or equivalent replacements that do not deviate from the concept of the invention are included within the scope of protection of the present invention.

The experimental materials and reagents used in the following examples can be obtained from commercial sources, prepared according to the prior art methods, or prepared according to methods similar to those disclosed in this application unless otherwise specified. The known compounds in the examples can be purchased commercially, prepared by the prior art methods, or are determined by comparison with data and/or properties of corresponding compounds in the prior art.

### Example 1

The fungus HDN16-608 (Penicillium guanacastense, preserved on Apr. 20, 2022, at the China Center for Type Culture Collection (CCTCC), CCTCC No: M 2022437) was first cultured on PDA solid slant medium (200 g/L potato, 20 g/L glucose, 20 g/L agar) in a 28° C. incubator for 6 days, then re-inoculated into PDB medium (200 g/L potato, 20 g/L glucose) for shaking fermentation for 12 days (28° C., 180 rpm). After filtration, the mycelium was crushed and extracted three times with methanol. The methanol was removed under reduced pressure, and the residue was extracted three times with ethyl acetate. The fermentation supernatant was extracted three times with an equal amount of ethyl acetate. All ethyl acetate phases were combined and concentrated under reduced pressure to obtain a crude extract. The above crude extract was initially separated by C-18 ODS column chromatography, gradient elution was performed with acetonitrile/water (80:20) as the mobile phase. Finally, reverse-phase semi-preparative high-performance liquid chromatography was used for separation and purification to obtain the following compound 2307 (acetonitrile/water, 85:15, YMC-Pack ODS-A, 10×250 mm, 5 μm, 3 mL/min, t_{R}=30 min):

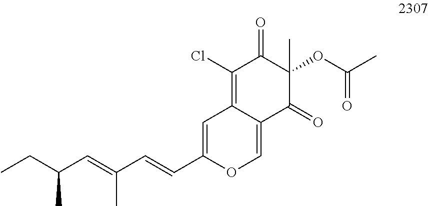 [SMILES: CC[C@H](C)/C=C(C)/C=C/C1=CC2=C(Cl)C(=O)[C@](C)(OC(C)=O)C(=O)C2=CO1]

### Example 2

Some I-I compounds can be obtained from compound 2307 through chemical transformation. The reaction process is as follows:

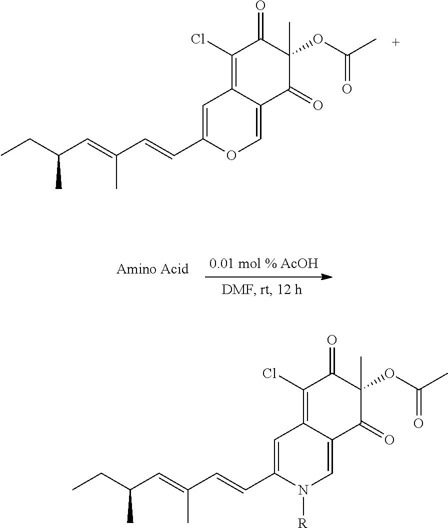

The specific implementation steps are as follows: Dissolve compound 2307 (10 mg) in 3 mL of N,N-dimethylformamide (DMF), add 3.0 eq of the corresponding amino acid (see the table below) and 0.01 mol % acetic acid, stir at room temperature for 12 h, filter, evaporate, and prepare by HPLC (YMC-Pack ODS-A, 10×250 mm, 5 μm, 3 mL/min) to obtain the corresponding product (see the table below).

| Product | | Amino acids used in the |
|---|---|---|
| number | R structure in the product | reaction |
| 1996 | —CH₂COOH | NH₂—CH₂COOH |
| 1998 | 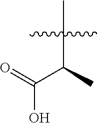 [SMILES: *[C@@H](C)C(=O)O] | 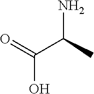 |
| 1999 | 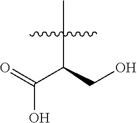 [SMILES: *[C@@H](CO)C(=O)O] | 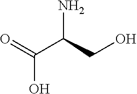 |
| 2000 | 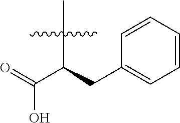 [SMILES: *[C@@H](Cc1ccccc1)C(=O)O] | 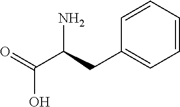 [SMILES: N[C@@H](Cc1ccccc1)C(=O)O] |
| 1997 | 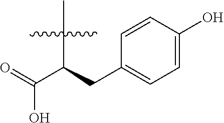 [SMILES: *[C@@H](Cc1ccc(O)cc1)C(=O)O] | 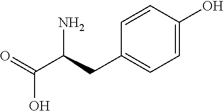 [SMILES: N[C@@H](Cc1ccc(O)cc1)C(=O)O] |
| 2001 | 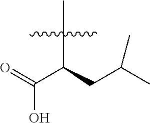 [SMILES: *[C@@H](CC(C)C)C(=O)O] | 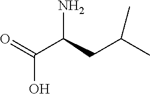 |
| 2309 | 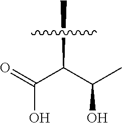 | 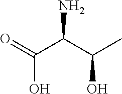 |
| Compound | | |
| number | Preparation gradient | Retention time |
| 2307 | MeCN:H₂O═85:15 | 30 min |
| 2308 | MeCN:H₂O═40:60 | 16 min |
| 1996 | MeCN:H₂O═40:60 | 24 min |
| 1998 | MeCN:H₂O═50:50 | 32 min |
| 1999 | MeCN:H₂O═50:50 | 30 min |
| 2000 | MeCN:H₂O═65:35 | 18 min |
| 1997 | MeCN:H₂O═50:50 | 27 min |
| 2001 | MeCN:H₂O═70:30 | 19 min |
| 2309 | MeCN:H₂O═50:50 | 35 min |

Compound 1996, red powder; HR-ESI-MS m/z: 448.1514 [M + H]⁺ (calculated for C₂₃H₂₆ClNO₆, 448.1521); ¹H and ¹³C NMR data are shown in Tables 1-1 and 1-2.
Compound 1998, red powder; HR-ESI-MS m/z: 462.1666 [M + H]⁺ (calculated for C₂₄H₂₉ClNO₆, 462.1678); ¹H and ¹³C NMR data are shown in Tables 1-1 and 1-2.
Compound 1999, red powder; HR-ESI-MS m/z: 478.1627 [M + H]⁺ (calculated for C₂₄H₃₀ClNO₇, 478.1627); ¹H and ¹³C NMR data are shown in Tables 1-1 and 1-2.
Compound 2000, red powder; HR-ESI-MS m/z: 536.1918 [M + H]⁺ (calculated for C₃₀H₃₃ClNO₆, 536.1916); ¹H and ¹³C NMR data are shown in Tables 1-1 and 1-2.
Compound 1997, red powder; HR-ESI-MS m/z: 554.1937 [M + H]⁺, calculated for C₃₀H₃₃ClNO₇, 554.1940); ¹H and ¹³C NMR data are shown in Tables 1-1 and 1-2.
Compound 2001, red powder; HR-ESI-MS m/z: 504.2147 [M + H]⁺ (calculated for C₂₇H₃₅ClNO₆, 504.2147); ¹H and ¹³C NMR data are shown in Tables 1-1 and 1-2.
Compound 2309, red powder; HR-ESI-MS m/z: 492.1775 [M + H]⁺ (calculated for C₂₅H₃₁ClNO₇, 492.1784); ¹H and ¹³C NMR data are shown in Tables 1-1 and 1-2.

TABLE 1-1
¹H NMR (500 MHz) data of compounds
2307 and 2308 in DMSO-d₆.

| | |
|---|---|
| 2307 | 2308 |

| No. | δ_{C}, type | δ_{H} (J in Hz) | δ_{C}, type | δ_{H} (J in Hz) |
|---|---|---|---|---|
| 1 | 154.9 CH | 8.34, s | 143.9, CH | 8.09, s |
| 3 | 158.8, C | | 149.6, C | |
| 4 | 107.7, CH | 6.94, s | 110.1, CH | 6.95, s |
| 4a | 114.2, C | | 145.1, C | |
| 5 | 109.0, C | | 99.5, C | |
| 6 | 185.3, C | | 182.7, C | |
| 7 | 85.2, C | | 85.3, C | |
| 8 | 191.6, C | | 193.5, C | |
| 8a | 139.8, C | | 113.8, C | |
| 9 | 117.3, CH | 6.54, d | 117.0, CH | 6.52, d |
| | | (15.7) | | (15.7) |
| 10 | 142.2, CH | 7.07, d | 144.2, CH | 7.11, d |
| | | (15.7) | | (15.7) |
| 11 | 114.2, C | | 132.8, C | |
| 12 | 148.1, CH | 5.77, d | 146.9, CH | 5.83, d |
| | | (9.7) | | (9.7) |
| 13 | 34.9, CH | 2.48, m | 34.7, CH | 2.48, m |
| 14 | 29.9, CH₂ | 1.29-1.39, m | 30.0, CH₂ | 1.28-1.34, m |
| 15 | 12.2, CH₃ | 0.82, t | 12.3, CH₃ | 0.84, t |
| | | (7.4) | | (7.4) |
| 16 | 20.5, CH₃ | 0.96, d | 20.6, CH₃ | 0.97, d |
| | | (6.6) | | (6.6) |
| 17 | 12.6, CH₃ | 1.82, s | 12.9, CH₃ | 1.84, s |
| 18 | 22.6, CH₃ | 1.49, s | 23.6, CH₃ | 1.30, s |
| 19 | 170.0, C | | 169.6, C | |
| 20 | 20.3, CH₃ | 2.10, s | 20.5, CH₃ | 2.06, s |
| 21 | | | 56.1, CH₂ | 3.65, s |
| 22 | | | 59.9, CH₂ | 4.20, s |

TABLE 1-2
¹H NMR (500 MHz) data of compounds in DMSO-d₆.

| No. | 1996 | 1998 | 1999 | 2000 | 1997 | 2001 | 2309 |
|---|---|---|---|---|---|---|---|
| 1 | 8.21, s | 8.05, s | 8.25, s | 8.06, brs | 8.06, brs | 8.07, s | 8.47, s |
| 4 | 6.95, s | 6.90, s | 6.86, s | — | — | 6.89, s | 6.91, s |
| 9 | 6.27, d | 6.33, d | 6.39, d | — | — | 7.07, d | 6.55, d |
| | (15.7) | (15.7) | (15.7) | | | | (15.7) |
| 10 | 7.11, d | 7.07, d | 7.01, d | — | — | 6.34, d | 7.05, d |
| | (15.7) | (15.7) | (15.7) | | | | (15.7) |
| 12 | 5.83, d | 5.81, d | 5.79, d | 5.81, d | 5.81, d | 5.80, d | 5.75, d |
| | (9.7) | (9.7) | (9.7) | (9.7) | (9.7) | (9.7) | (9.7) |
| 13 | 2.48, m | 2.48, m | 2.48, m | 2.48, m | 2.48, m | 2.48, m | 2.52, m |
| 14 | 1.28-1.38, m | 1.28-1.38, m | 1.28-1.38, m | 1.28-1.38, | 1.28-1.38, m | 1.28-1.38, m | 1.28-1.38, m |
| 15 | 0.83, t | 0.84, t | 0.83, t | 0.83, t | 0.83, t | 0.81, t | 0.87, t |
| | (7.4) | (7.4) | (7.4) | (7.4) | (7.4) | (7.4) | (7.4) |
| 16 | 0.97, d | 0.97, d | 0.97, d | 0.95, d | 0.95, d | 0.97, d | 1.00, d |
| | (6.6) | (6.6) | (6.6) | (6.6) | (6.6) | (6.6) | (6.6) |
| 17 | 1.79, s | 1.81, s | 1.81, s | 1.76, s | 1.76, s | 1.81, s | 1.90, s |
| 18 | 1.41, s | 1.41, s | 1.41, s | 1.31, s | 1.31, s | 1.41, s | 1.46, s |
| 20 | 2.07, s | 2.07, s | 2.06, s | 2.06, s | 2.06, s | 2.07, s | 2.04, s |
| 21 | 5.07, s | 5.40, q | 5.28, brs | 5.40, q | 5.40, q | 5.24, brs | 5.30, q |
| | | (7.0) | | (7.0) | (7.0) | | (7.0) |
| 22 | | | | 1.68, d | 1.68, d | | 4.80, brs |
| | | | | (7.0) | (7.0) | | |
| 23 | | 1.68, d | 3.99-4.11, m | 3.52, brs | 3.52, brs | 2.00, brs | |
| | | (7.0) | | | | | |
| 24 | | | | | | 1.45, m | 1.26, d |
| | | | | | | | (6.0) |
| 25 | | | | 7.13, m | 7.13, m | 0.87, d | |
| | | | | | | (6.3) | |
| 26 | | | | 7.24, m | 7.24, m | 0.87, d | |
| | | | | | | (6.3) | |
| 27 | | | | 7.18, m | | | |
| 28 | | | | 7.24, m | 7.24, m | | |
| 29 | | | | 7.13, m | 7.13, m | | |

TABLE 1-3
¹³C NMR (125 MHz) data of compounds in DMSO-d₆.

| No. | 1996 | 1998 | 1999 | 2000 | 1997 | 2001 | 2309 |
|---|---|---|---|---|---|---|---|
| 1 | 144.1, CH | 144.1, CH | 142.4, CH | 144.0, CH | 144.1, CH | —, CH | 142.2, CH |
| 3 | 149.5, C | 149.7, C | 149.7, C | —, C | —, C | —, C | 150.5, C |
| 4 | 119.6, CH | 110.6, CH | 110.6, CH | 110.1, CH | 110.1, CH | 110.6, CH | 111.1, CH |
| 4a | 114.0, C | 114.2, C | 113.7, C | —, C | —, C | 114.3, C | 113.8, C |
| 5 | 100.4, C | 100.6, C | 100.3, C | —, C | —, C | 100.9, C | 101.7, C |
| 6 | 183.1, C | 183.3, C | 183.2, C | 183.1, C | 183.1, C | 183.3, C | 183.8, C |
| 7 | 85.2, C | 85.2, C | 85.2, C | 85.2, C | 85.2, C | 85.2, C | 85.4, C |
| 8 | 193.3, C | 193.2, C | 193.4, C | 193.0, C | 193.1, C | 193.2, C | 192.8, C |
| 8a | 144.7, C | 144.4, C | 144.5, C | —, C | —, C | —, C | 144.0, C |
| 9 | 116.0, CH | 116.5, CH | 116.9, CH | 116.4, CH | 116.4, CH | 116.6, CH | 116.6, CH |
| 10 | 144.8, CH | 144.6, CH | 144.6, CH | 144.6, CH | 144.5, CH | 144.2, CH | 144.8, CH |
| 11 | 132.7, C | 132.7, C | 132.8, C | 132.7, C | 132.7, C | 132.6, C | 132.5, C |
| 12 | 147.4, CH | 147.1, CH | 146.7, CH | 147.2, CH | 147.2, CH | 147.1, CH | 146.2, CH |
| 13 | 34.7, CH | 34.7, CH | 34.7, CH | 34.7, CH | 34.7, CH | 34.7, CH | 34.7, CH |
| 14 | 30.0, CH₂ | 30.0, CH₂ | 30.0, CH₂ | 30.0, CH₂ | 30.0, CH₂ | 30.0, CH₂ | 29.8, CH₂ |
| 15 | 12.3, CH₃ | 12.3, CH₃ | 12.3, CH₃ | 12.3, CH₃ | 12.3, CH₃ | 12.2, CH₃ | 11.5, CH₃ |
| 16 | 20.6, CH₃ | 20.6, CH₃ | 20.6, CH₃ | 20.6, CH₃ | 20.6, CH₃ | 20.5, CH₃ | 19.4, CH₃ |
| 17 | 12.7, CH₃ | 12.7, CH₃ | 12.7, CH₃ | 12.7, CH₃ | 12.7, CH₃ | 12.7, CH₃ | 11.9, CH₃ |
| 18 | 23.5, CH₃ | 23.4, CH₃ | 23.4, CH₃ | 20.5, CH₃ | 20.5, CH₃ | 23.4, CH₃ | 22.7 CH₃ |
| 19 | 169.7, C | 169.7, C | 169.7, C | 170.2, C | 170.3, C | 169.7, C | 168.7, C |
| 20 | 20.5, CH₃ | 20.6, CH₃ | 20.6, CH₃ | 23.4, CH₃ | 23.4, CH₃ | 20.6, CH₃ | 19.7, CH₃ |
| 21 | 54.7, CH₂ | 60.4, CH | 44.5, CH | 44.6, CH | 44.6, CH | —, C | 66.9, CH |
| 22 | 169.5, C | 171.4, C | 16934, C | 169.6, C | 169.6, C | 171.1, C | 168.9, C |
| 23 | | 17.1, CH₃ | 61.2, CH₂ | 36.9, CH₂ | —, CH₂ | 39.8, CH₂ | 67.4, CH |
| 24 | | | | 36.2, C | 36.2, C | 25.0, CH | 19.7, CH₃ |
| 25 | | | | 129.5, CH | 130.5, CH | 21.8, CH₃ | |
| 26 | | | | 129.0, CH | 115.8, CH | 23.0, CH₃ | |
| 27 | | | | 127.5, CH | 156.8, C | | |
| 28 | | | | 129.0, CH | 115.8, CH | | |
| 29 | | | | 129.5, CH | 130.5, CH | | |

### Example 3

The fungus HDN151398 (Chaetomium globosum, preserved at the China Center for Type Culture Collection (CCTCC), CCTCC No: M 2022438, on Apr. 20, 2022), was first cultivated on PDA solid slant medium in 28° C. incubator for 4 days. It was then inoculated into Fungal Liquid Fermentation Medium No. 2 and fermented at room temperature (20° C.) for 30 days. The fermentation broth was filtered, and the mycelium was broken and extracted three times with methanol. The methanol was removed under reduced pressure, and the residue was extracted three times with ethyl acetate. The fermentation supernatant was also extracted three times with an equal amount of ethyl acetate. All ethyl acetate phases were combined and concentrated under reduced pressure to obtain a crude extract. The above crude extract was initially separated by a C-18 ODS column chromatography, gradient elution was performed with acetonitrile/water as the mobile phase. Further separation was performed by a Sephadex LH20 gel column chromatography with methanol/water as the mobile phase. Finally, reverse-phase semi-preparative high-performance liquid chromatography (RP-HPLC) was used for separation and purification (YMC-Pack ODS-A, 10×250 mm, 5 μm, 3 mL/min) to obtain the following compounds:

| Compound | | Retention |
|---|---|---|
| number | Preparation gradient | time |
| 1778 | MeOH:H₂O = 45:55 | 40 min |
| 2318 | MeOH:H₂O = 70:30 | 28 min |
| 2320 | MeOH:H₂O = 70:30 | 36 min |
| 1779 | MeOH:H₂O = 70:30 | 27 min |
| 2316 | MeOH:H₂O = 65:35 | 34 min |
| 1775 | MeOH:H₂O = 70:30 | 34 min |
| 1780 | MeOH:H₂O = 70:30 | 26 min |

The composition of Fungus Medium No. 2:

| | | |
|---|---|---|
| Mannitol 2.0% | Yeast extract 0.3% | MgSO₄•7H₂O 0.03% |
| Monosodium | Glucose 1.0% | KH₂PO₄ 0.05% |
| Glutamate 1.0% | | |
| Maltose 2.0% | Corn steep | |
| | liquor 0.1% | |

Sodium glutamate can be replaced by _{L}-tryptophan or _{L}-histidine to produce compounds 2318 and 2320.

TABLE 1-4
¹H NMR (500 MHz) data of compounds
2318, 2320, 1778, and 1779.

| No. | 2318^{c} | 2320^{c} | 1778^{b} | 1779^{a} |
|---|---|---|---|---|
| 1 | 8.61, s | 8.99, s | 7.76, s | 7.81, s |
| 4 | 6.42, s | 6.58, s | 6.90, s | 6.72 |
| 9 | 6.14, d | 6.34, d | 6.46, d | 6.17, d |
| | (12.8) | (15.7) | (14.2) | (15.4) |
| 10 | 6.01, dd | 6.25, m | 6.41, m | 6.32, dd |
| | (5.9, 12.8) | | | (7.6, 15.4) |
| 11 | 2.06, m | 2.26, m | 2.35, m | 2.28, m |
| 12 | 1.26, m | 1.40, m | 1.51, m | 1.44, m |
| 13 | 0.76, t | 0.85, t | 0.96, t | 0.91, t |
| | (6.2) | (7.4) | (7.5) | (7.5) |
| 14 | 0.98, d | 1.03, d | 1.13, d | 1.08, d |
| | (5.6) | (6.7) | (6.7) | (6.7) |
| 15 | 1.43, s | 1.60, s | 1.69, s | 1.69, s |
| 2′ | 5.53, dd | 5.48, t | 5.26, m | 5.05, t |
| | (4.1, 8.5) | (7.7) | | (7.3) |
| 3′ | 3.48, m | 3.49, m | 2.29, m | 2.47, m |
| | 3.68, dd | | 2.57, m | 2.30, m |
| | (4.1, 12.8) | | | |
| 4′ | | | 2.44, t | 2.42, m |
| | | | (6.6) | |
| | | | 2.49, m | 2.56, m |
| 5′ | | 7.34, s | | |
| 6′ | 7.49, d | | | 3.67, s |
| | (6.6) | | | |
| 7′ | 6.91, t | 7.41, s | | 3.77, s |
| | (6.2) | | | |
| 8′ | 7.03, t | | | |
| | (6.1) | | | |
| 9′ | 7.29, d | | | |
| | (6.8) | | | |
| 11′ | 10.94, s | | | |
| 12′ | 7.14, d | | | |
| | (1.9) | | | |
| 4″ | 3.56, m | | | |
| 5″ | 3.62, m | 6.65, d | 6.64, q | 6.55, q |
| | | (6.8) | (6.9) | (6.3) |
| 6″ | 0.89, d | 1.82, d | 1.89, d | 1.87, d |
| | (5.2) | (6.8) | (7.0) | (6.9) |
| 7″ | 0.92, d | 1.77, s | 1.87, s | 1.83, s |
| | (5.6) | | | |

^{a}Measured in CDCl₃; ^{b}Measured in methanol-d₄; ^{c}Measured in DMSO-d₆.

TABLE 1-5
¹H NMR (500 MHz) data of compounds
2316, 1775, and 1780 in CDCl₃.

| No. | 2316 ^{a} | 1775 ^{a} | 1780 ^{a} |
|---|---|---|---|
| 1 | 7.27, s | 7.28, s | 7.90, s |
| 4 | 6.57, s | 6.55, s | 6.53, s |
| 8 | 2.98, d (10.0) | 2.98, d (10.0) | 4.22, s |
| 9 | 6.15, d (15.0) | 6.06, d (15.5) | 6.04, d (15.5) |
| 10 | 6.61, dd (15.0, 6.2) | 6.52, dd (15.5, 6.5) | 6.52, dd (15.5, 6.5) |
| 11 | 2.45, m | 2.26, m | 2.25, m |
| 12 | 3.81, brs | 1.43, m | 1.43, m |
| 13 | 1.20, d (6.2) | 0.91, t (7.5) | 0.90, t (7.6) |
| 14 | 1.13, d (6.2) | 1.08, d (6.5) | 1.08, d (6.5) |
| 15 | 1.40, s | 1.40, s | 1.72, s |
| 2′ | 3.06, d (10.0) | 3.08, d (10.0) | |
| 4′ | 1.90, m | 1.90, m | 2.58, m |
| 5′ | 4.30, m | 4.30, m | 4.29, m |
| 6′ | 1.41, d (6.5) | 1.41, d (6.2) | 1.40, d (7.0) |
| 7′ | 1.13, d (6.2) | 1.13, d (6.5) | 1.24, d (7.0) |

^{a} Measured in CDCl₃

### Biological Experiment

### 1. Experimental Materials

Fetal bovine serum and horse serum, purchased from GIBCO, USA. α-MEM medium, purchased from HyClone, USA. IMDM medium and DMEM medium, purchased from Geno Biopharmaceutical Technology Co., Ltd., China. NK92 cells, purchased from ATCC, USA. NCI-H1975, K562, HepG2, MDA-MB-231, NCI-H446, A2058, and B16F10 cells, purchased from the Shanghai Cell Bank, Chinese Academy of Sciences. Recombinant human interleukin-2 (rhIL-2), purchased from Peprotech, USA. Human granzyme B, perforin, and IFN-γ cytokine ELISA test kits, purchased from Dako Diagnostics, China. Lymphocyte separation solution, purchased from Solarbio Science & Technology Co., Ltd., China. CCK-8 test kit, purchased from Shanghai Yisheng Biotechnology Co., Ltd., China. CD107a antibody, CD56 antibody, CD3 antibody, NK1.1 antibody, and NKP46 antibody, purchased from BD Biosciences, USA. Lactate dehydrogenase cytotoxicity test kit, purchased from BioGend Biotech Co., Ltd., China. Experimental animals from Jinan Pengyue Experimental Animal Breeding Co., Ltd., China.

### II. Experimental Methods

### 1. Preparation and Treatment of Experimental Cells

### 1.1 Cell Culture

| Cell Line | Culture medium | Chinese name |
|---|---|---|
| NK-92 | α-MEM + 12.5% fetal | Human natural killer cells |
| | bovine serum + 12.5% | |
| | horse serum + 200U IL-2 | |
| K562 | IMEM + 10% fetal | Human chronic myeloid |
| | bovine serum | leukemia cells |
| NCI-H446 | DMEM + 10% fetal | Human small cell lung |
| | bovine serum | cancer cells |
| MDA-MB-231 | DMEM + 10% fetal | Human triple-negative |
| | bovine serum | breast cancer cells |
| Hep-G2 | DMEM + 10% fetal | Human hepatocellular |
| | bovine serum | carcinoma cell |
| NCI-H1975 | DMEM + 10% fetal | Human non-small cell lung |
| | bovine serum | adenocarcinoma cells |
| A2058 | DMEM + 10% fetal | Human skin melanoma |
| | bovine serum | cells |

### 1.2 Cell Thawing

Remove the frozen cells from the liquid nitrogen tank and quickly place them in a 37° C. water bath to thaw rapidly. Then, transfer the contents of the cryovial to a centrifuge tube containing 10 mL of a-MEM medium, centrifuge at 350 g for 5 min, and discard the supernatant. Add 5 mL of fresh a-MEM medium, mix by pipetting, and transfer the cell suspension to a culture flask. Place the flask in a 37° C., 5% CO₂ incubator for incubation.

### 1.3 Cell Passage

Collect the cell suspension in a centrifuge tube, centrifuge at 350 g for 5 min, and discard the supernatant. Add a-MEM complete medium again, mix well by pipetting, and distribute the cell suspension evenly into two culture flasks.

### 2. Effect of The Killing Function of Azaphilone Compounds on NK92 Cell

### 2.1 NK92 Cell Seeding and Drug Treatment in 24-Well Plates

Seed the NK92 cells obtained from 1.3 into a 24-well cell culture plate (referred to as a 24-well plate) at a concentration of 3×10⁵ cells/well, with 4 wells designated as blank or drug-treated. For the drug-treated wells, set the initial screening concentration to 100 nM. Add 900 μL of cell suspension and 100 μL of drug to each well. For the blank wells, add 100 μL of double-distilled water. Incubate the 24-well plate in a 37° C., 5% CO₂ incubator for 72 h. Replace the fresh culture medium and add drugs every 24 h during the incubation.

### 2.2 Tumor Target Cell Seeding in 96-Well Plates

Seed various types of tumor cells in logarithmic phase into a 96-well cell culture plate at a concentration of 5×10³ cells/well. Set up 3 replicate wells for each concentration and include wells for target cell spontaneous release and target cell maximum release. Incubate the 96-well plate overnight in a 37° C., 5% CO₂ incubator.

### 2.3 Adding NK92 Cells to 96-Well Plates According to the Set Target-to-Effector Ratio

Centrifuge and count the NK92 cells from 2.1 in the 24-well plate cultured for 72 h. Add the cells to the overnight-cultured tumor target cells according to the effector-to-target ratio of 5:1 (excluding wells for target cell spontaneous release and maximum release). Also, set up 3 effector cell control wells. Add 200 μL of cell suspension to each well, then centrifuge at 300 g, 25° C. for 5 min to allow sufficient contact between effector cells and target cells. Continue to place the 96 well plate in a 37° C., 5% CO₂ incubator and co-cultivate the NK92 and target cells for 5 h.

### 2.4 Detection of the Killing Ability of NK Cell Against Tumor Cells

Lactate dehydrogenase (LDH) assay kit (BioGend Biotech Co., Ltd.) is used to detect the killing effect of effector cells on target cells. Add LDH release reagent to the wells with target cell maximum release and incubate for 1 h. After 1 h, centrifuge at 500 g, 25° C. for 5 min. Transfer 120 μL of supernatant from each well to a new 96-well plate, add the prepared LDH detection solution (60 μL/well: 20 μL lactate solution, 20 μL 1×INT working solution, and 20 μL enzyme solution) in the dark, and mix well by pipetting. Incubate at room temperature in the dark for 20 min (wrapped in aluminum foil and shook slowly on a shaker). Measure the absorbance at 490 nm using a microplate reader and the measured absorbance value of each well should subtract the absorbance value of the background blank control well.

Cytotoxicity (%)=[(Experimental group−Effector cell control−Target cell spontaneous release)/(Target cell maximum release−Target cell spontaneous release)]×100.

| | | | | tumor killing |
|---|---|---|---|---|
| Sample | Molecular | Molecular | | rate by NK |
| number | formula | weight | Structure | activation |
| 2307 | C₂₁H₂₃ClO₅ | 390 | 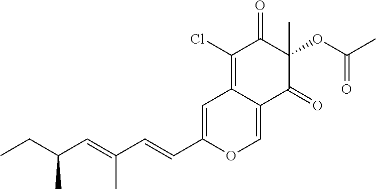 [SMILES: CC[C@H](C)/C=C(C)/C=C/C1=CC2=C(Cl)C(=O)[C@](C)(OC(C)=O)C(=O)C2=CO1] | 38.29% |
| 2308 | C₂₃H₂₈ClNO₅ | 433 | 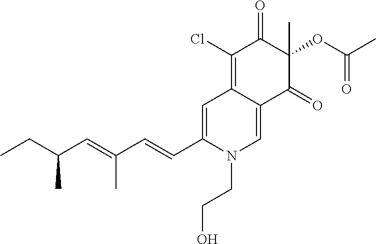 [SMILES: CC[C@H](C)/C=C(C)/C=C/C1=CC2=C(Cl)C(=O)[C@](C)(OC(C)=O)C(=O)C2=CN1CCO] | 8.11% |
| 1996 | C₂₃H₂₆ClNO₆ | 447 | 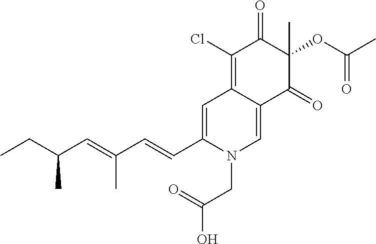 [SMILES: CC[C@H](C)/C=C(C)/C=C/C1=CC2=C(Cl)C(=O)[C@](C)(OC(C)=O)C(=O)C2=CN1CC(=O)O] | 6.12% |
| 1998 | C₂₄H₂₈ClNO₆ | 461 | 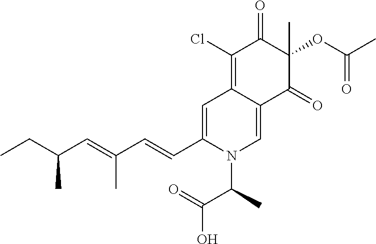 [SMILES: CC[C@H](C)/C=C(C)/C=C/C1=CC2=C(Cl)C(=O)[C@](C)(OC(C)=O)C(=O)C2=CN1[C@@H](C)C(=O)O] | 78.44% |
| 1999 | C₂₄H₂₈ClNO₇ | 477 | 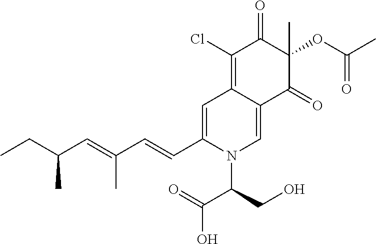 [SMILES: CC[C@H](C)/C=C(C)/C=C/C1=CC2=C(Cl)C(=O)[C@](C)(OC(C)=O)C(=O)C2=CN1[C@@H](CO)C(=O)O] | 58.29% |
| 2000 | C₃₀H₃₂ClNO₆ | 537 | 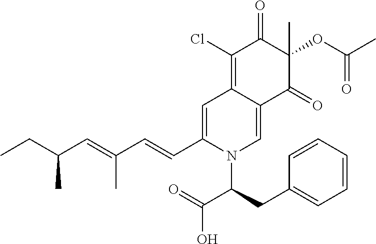 [SMILES: CC[C@H](C)/C=C(C)/C=C/C1=CC2=C(Cl)C(=O)[C@](C)(OC(C)=O)C(=O)C2=CN1[C@@H](Cc1ccccc1)C(=O)O] | 65.25% |
| 1997 | C₃₀H₃₂ClNO₇ | 553 | 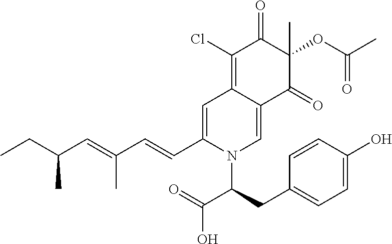 [SMILES: CC[C@H](C)/C=C(C)/C=C/C1=CC2=C(Cl)C(=O)[C@](C)(OC(C)=O)C(=O)C2=CN1[C@@H](Cc1ccc(O)cc1)C(=O)O] | 29.52% |
| 2001 | C₂₇H₃₄ClNO₆ | 503 | 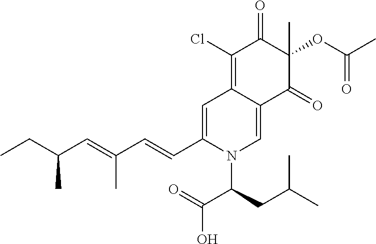 [SMILES: CC[C@H](C)/C=C(C)/C=C/C1=CC2=C(Cl)C(=O)[C@](C)(OC(C)=O)C(=O)C2=CN1[C@@H](CC(C)C)C(=O)O] | 18.73% |
| 2309 | C₂₅H₃₀ClNO₇ | 491 | 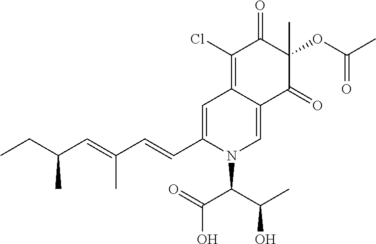 [SMILES: CC[C@H](C)/C=C(C)/C=C/C1=CC2=C(Cl)C(=O)[C@](C)(OC(C)=O)C(=O)C2=CN1[C@H](C(=O)O)[C@@H](C)O] | 28.34% |
| 1778 | C₂₈H₃₀ClNO₈ | 543 | 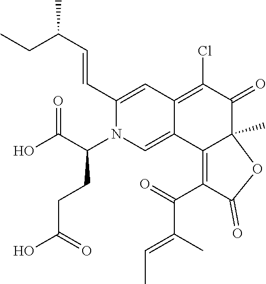 [SMILES: C/C=C(\C)C(=O)C1=C2C3=CN([C@@H](CCC(=O)O)C(=O)O)C(/C=C/[C@@H](C)CC)=CC3=C(Cl)C(=O)[C@@]2(C)OC1=O] | 75.20% |
| 1779 | C₃₀H₃₄ClNO₈ | 571 | 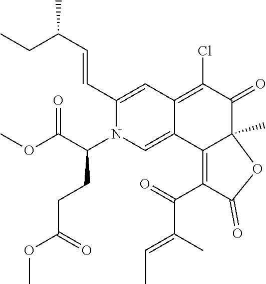 [SMILES: C/C=C(\C)C(=O)C1=C2C3=CN([C@@H](CCC(=O)OC)C(=O)OC)C(/C=C/[C@@H](C)CC)=CC3=C(Cl)C(=O)[C@@]2(C)OC1=O] | 21.96% |
| 2318 | C₃₄H₃₃ClN2O₆ | 618 | 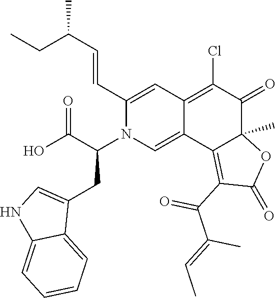 [SMILES: C/C=C(\C)C(=O)C1=C2C3=CN([C@@H](Cc4c[nH]c5ccccc45)C(=O)O)C(/C=C/[C@@H](C)CC)=CC3=C(Cl)C(=O)[C@@]2(C)OC1=O] | 35.85% |
| 2320 | C₂₉H₃₀ClN3O₆ | 551 | 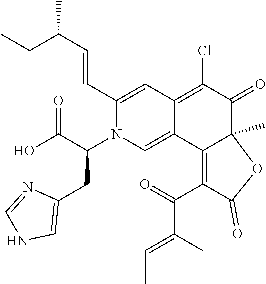 [SMILES: C/C=C(\C)C(=O)C1=C2C3=CN([C@@H](Cc4c[nH]cn4)C(=O)O)C(/C=C/[C@@H](C)CC)=CC3=C(Cl)C(=O)[C@@]2(C)OC1=O] | 22.18% |
| 2316 | C₂₃H₂₇ClO₆ | 434 | 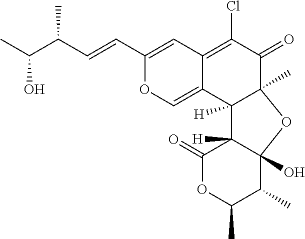 [SMILES: C[C@H](/C=C/C1=CC2=C(Cl)C(=O)[C@@]3(C)O[C@]4(O)[C@H](C)[C@@H](C)OC(=O)[C@@H]4[C@H]3C2=CO1)[C@@H](C)O] | 31.09% |
| 1775 | C₂₃H₂₇ClO₇ | 450 | [symbol] | 0.58% |
| 1780 | C₂₃H₂₇ClO₅ | 418 | [symbol] | 8.53% |

[symbol]

indicates data missing or illegible when filed

From above table, it can be seen that the compounds listed have significant activation effects on NK92 cells, especially compound 1778, which has a very strong activation effect.

3. Determination of NK Cell Proliferation Rate Using CCK-8 after Treatment with 1778
3.1 Preparation of Compound Concentration Gradient: Dilute the azaphilone compound 1778 with double-distilled water to set up 5 concentrations, with final concentrations of 1, 10, 100, 1000, and 10000 nM in the wells.

### 3.2 NK92 Cell Seeding and Drug Treatment in 96-Well Plates

Seed NK92 cells obtained from 1.3 into 96-well cell culture plate (referred to as a 96-well plate) at a concentration of 1×10⁴ cells/well, 90 pL/well. Set up 5 replicates for each compound concentration (also need to set a blank group, that is, double-distilled water replaces the compound), and seal wells with PBS. Incubate in a 37° C., 5% CO₂ incubator for 72 h.

### 3.3 CCK-8 Assay

Remove the cell culture plates from 2.2 and add 10 μL of CCK-8 solution (Shanghai Yisheng Biotechnology Co., Ltd.) to each well, avoiding bubbles. After adding CCK-8 solution, incubate the 96-well cell culture plates in a 37° C., 5% CO₂ incubator for 2.5 h. Measure the absorbance at 450 nm using a microplate reader to compare the proliferation level of NK92 cells. Results are shown in FIG. 1.

4. Detection of The Killing Ability of NK Cell against Tumor Cells After Treatment with 1778

Set up three concentrations of compound 1778, namely 1, 10, and 100 nM. All other procedures are the same as in section 2.

5. Detection of NK Cell Activation Factor Release after Treatment with 1778

NK cells release granzyme B and perforin to kill tumor cells upon activation, while release IFN-γ to regulate the mechanism of antitumor immunity.

5.1 Pre-treatment of NK92 Cells with Compound 1778: Seed NK92 cells into a 24-well plate and treat with drug as described in section 3.1.

### 5.2 Seeding Target Cells NCI-H1975 in 96-Well Plates

Seed logarithmic phase NCI-H1975 cells into 96-well plates at a concentration of 1×10⁴ cells/well. Set up 3 replicates for each concentration and control wells for target cell. Incubate the 96-well plate overnight in a 37° C., 5% CO₂ incubator.

5.3 Co-Culture of NK92 Cells with Target Cells NCI-H1975 for Detection

According to the effector-to-target ratio of 5:1, add NK92 cells to NCI-H1975, co-cultivate for over 6 h and centrifuge (500 g, 25° C.). Then collect supernatant (use within 24 h). Use ELISA kits (BD Biosciences, USA) to detect the release of 3 activating cytokines granzyme B (FIG. 3), perforin (FIG. 4), and IFN-γ (FIG. 5).

6. Detection of Primary NK Cell Activation in Peripheral Blood PBMCs from Healthy Donors and Pleural Effusion from Tumor Patients After Treatment with 1778

CD107 is expressed on the surface of NK cells released by the activator. Therefore, the activation of NK cells can be reflected by detecting the content of CD107 expressed by cell membrane.

6.1 Collect peripheral blood from normal individuals and pleural effusion from lung cancer patients using sterile anticoagulant tubes, and dilute with an equal volume of sterile PBS. Take a 50 ml centrifuge tube and absorb a certain volume of lymphocyte separation solution (Solarbio Science & Technology Co., Ltd.). Slowly add diluted peripheral blood and pleural effusion in a volume ratio of 4:3. Centrifuge at 400 g at room temperature for 30 min (Adjust the lifting and deceleration speed of the centrifuge to rise 1 or down 1).
6.2 Carefully absorb the white lymphocyte layer in the middle and place it in PBS (at least three times the volume of the absorbed cells). Overturn and blending, and wash the cells. Centrifuge at 100 g at room temperature for 10 min. Discard the supernatant, resuspend the cell pellet in 10 ml PBS, and gently mix. Wash off residual Ficoll, then centrifuge again at 100 g for 10 min. Discard the supernatant, resuspend the cell pellet in RPMI-1640 complete medium, and continue culture for 3 days with RPMI-1640 complete medium containing 10 ng/ml IL-2 to stimulate primary NK cell expansion and activation. After activation, seed the primary cells in a 24-well plate at a concentration of 3×10⁵ cells/well. Set up blank wells and drug-treated wells with compound 1778 at concentrations of 1, 10, and 100 nM, and incubate for 72 h.
6.3 Collect target cell suspension in a 1.5 mL EP tube by using a pipette, centrifuge at 1000 r/min for 5 min. Discard the supernatant, add a clean NK culture medium to resuspend cells, and count the treated NK cells and K562 target cells by using a blood cell counting plate respectively. According to an effector-to-target ratio of 5:1, add to the 24-well plate with a volume of 1 mL per well.
6.4 Co-culture for 4 h, then centrifuge and discard the supernatant. Add 100 μL of pre-cooled PBS (containing 1% FBS) to each tube. Add CD3, CD56, and CD107a antibodies (BD Biosciences, USA) and stain in the dark for 40 min, wash twice with PBS, add 400 μL PBS (containing 1% FBS) to each tube, resuspend, filter and transfer to flow cytometry tubes for on-machine analysis. The detection results were analyzed using FlowJo software. The results are shown in FIGS. 6 and 7.

### 7. In Vivo Evaluation of 1778 on Melanoma Lung Metastases in C57BL/6 Mice

To investigate whether 1778 works in vivo, we used a melanoma lung metastasis model.

Mouse B16F10 melanoma cells were intravenously injected into mice, and then 1778 was administered for efficacy evaluation. B116F10 cells are highly metastatic and lack MHC class-I expression, and it has been previously reported that the metastasis of B116F10 cells in vivo is mainly controlled by NK cells.

7.1 Inject 200,000 B116F10 cells via the tail vein into each C57BL/6J mouse.
7.2 A daily intraperitoneal injection of 1778 at a dose of 25 mg/kg (diluted with PBS solution to a final volume of 200 μL). The control group was injected intraperitoneally with an equivalent volume of PBS (with the same concentration of solvent DMSO). Weigh the mice every other day to monitor the impact on body weight. After 14 days of drug administration, euthanize the mice, collect lung tissue, and fix it in tissue fixative. Count and statistically analyze the number of melanoma colonies in the lungs. The detection results are shown in FIG. 8.

### 7. Statistical Analysis

Analyze data using SPSS 16.0 software, presenting as mean±standard deviation. Compare groups using One-way ANOVA, with P<0.05 considered statistically significant.

As shown in FIG. 1, compound 1778 has a weak proliferative effect on NK92 cells. However, compound 1778 significantly enhances the ability of NK92 cells to kill various types of tumor cells, as shown in FIG. 2. Compound 1778 promotes the releases of cytokines such as granzyme B (FIG. 3), perforin (FIG. 4), and IFN-γ (FIG. 5) after NK92 cell activation. Following treatment with compound 1778, there was a significant increase in the expression of CD107 in primary NK cells isolated from the peripheral blood of healthy individuals (FIG. 6) and from the pleural fluid of lung cancer patients (FIG. 7), indicating that 1778 promotes activation and degranulation of human NK cells. As shown in FIG. 8, in vivo administration of 1778 significantly reduced the number of lung metastases in mice, indicating that 1778 has an inhibitory effect on melanoma lung metastasis.

The above-described examples are merely preferred embodiments of the present invention and do not serve to limit the invention. Any modifications, equivalent substitutions, or improvements made within the spirit and principle of the invention are considered to be within the scope of the invention's protection.

1. Use of compound of formula I or its pharmaceutically acceptable salts, stereoisomers, 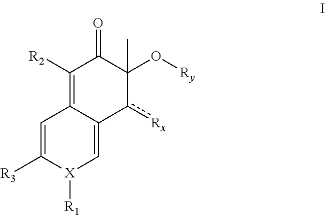
wherein
R₁ is absent, HOC₁-C₆-alkyl-, —C₁-C₆-alkyl-COOR₇, wherein the C₁-C₆-alkyl is optionally substituted with 1, 2, or 3 groups independently selected from: —OH, C₁-C₆-alkyl-, C₁-C₆-alkyl optionally substituted with OH, C₆-C₁₀-aryl optionally substituted with OH, C₆-C₁₀-aryl-C₁-C₆-alkyl optionally substituted with OH, —C₁-C₆-alkyl-COOR₈, 5-10 membered heteroaryl optionally substituted with OH, and 5-10 membered heteroaryl-C₁-C₆-alkyl- optionally substituted with OH;
X is O or N; provided that when X is O, R₁ is absent;
R₂ is a halogen;
R₃ is selected from C₁-C₆-alkyl-, C₂-C₆-alkenyl-, which is optionally substituted with one, two, or three groups selected from C₁-C₃-alkyl and —OH; [symbol]
represents a single or double bond;
Rₓ is =O and R_{y} is R₄;
or Rₓ and R_{y} together represent the structure: 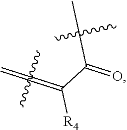
wherein the carbonyl carbon is connected to the O atom shown in formula I; or 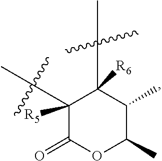
wherein the carbon carrying R₆ is connected to the O atom shown in formula I;
R₄ is selected from —CO—C₁-C₆-alkyl and —CO—C₂-C₆-alkenyl;
R₅ and R₆ are independently H or OH;
or R₅ and R₆ are linked together to form a double bond between the two carbon atoms to which they are attached;
R₇ and R₈ are independently selected from H and C₁-C₆-alkyl;
in the preparation of a medicament for treating diseases treatable by NK cell activation.

2. The use according to claim 1, wherein, in the compound of formula I, R₁ is absent, HOC₁-C₃-alkyl-, —C₁-C₃-alkyl-COOR₇, wherein the C₁-C₃-alkyl is optionally substituted with one group independently selected from: —OH, C₁-C₃-alkyl-, C₁-C₃-alkyl- optionally substituted with OH, phenyl- optionally substituted with OH, phenyl-C₁-C₃-alkyl- optionally substituted with OH, —C₁-C₃-alkyl-COOR₈, 5-10 membered heteroaryl containing one or two heteroatoms selected from N, O, or S optionally substituted with OH, and 5-10 membered heteroaryl-C₁-C₃-alkyl- containing one or two heteroatoms selected from N, O, or S optionally substituted with OH; preferably, the 5-10 membered heteroaryl is selected from: indolyl, isoindolyl, pyrazolyl, imidazolyl, pyridyl, and oxazolyl;
R₃ is selected from C₄-C₆-alkyl, C₄-C₆-alkenyl, which is optionally substituted with one, two, or three C₁-C₃-alkyl or one —OH;
R₇ and R₈ are independently selected from H and C₁-C₃-alkyl.

3. The use according to claim 1 or 2, wherein, in the compound of formula I, R₁ is absent, —CH₂CH₂OH, —CH₂COOH, 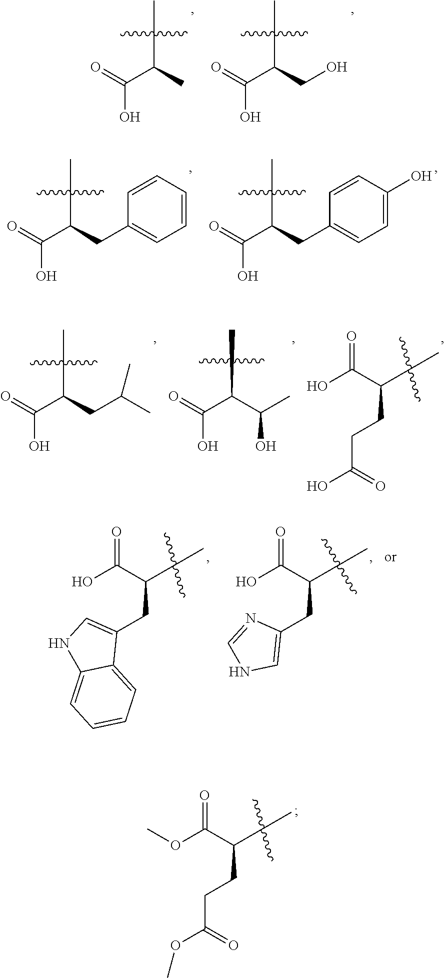
X is O or N; provided that when X is O, R₁ is absent;
R₂ is Cl;
R₃ is selected from 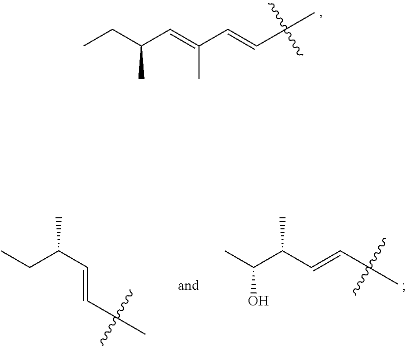
R₄ is CH₃CO— or 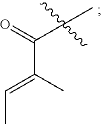 [SMILES: *C(=O)/C(C)=C/C]
R₅ is H;
R₆ is OH;
or R₅ and R₆ are linked together to form a double bond between the two carbon atoms to which they are attached.

4. The use according to any one of claims 1-3, wherein the compound of formula I has the following structure of formula I-I, I-II, or I-III: 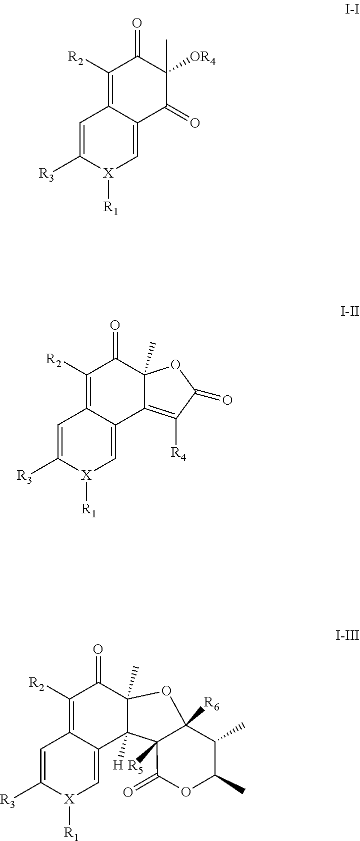
wherein R₁, R₂, R₃, R₄, R₅, R₆, X are as defined in one of claims 1-3.

5. The use according to any one of claims 1, 2, and 4, wherein the compound of formula I has the following structure of formula I-I, I-II, or I-III: 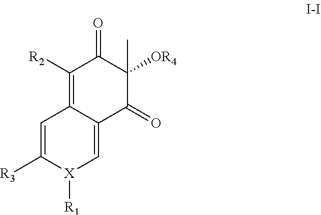
wherein R₁ is absent, HOC₁-C₃-alkyl- or —C₁-C₃-alkyl-COOH, wherein the C₁-C₃-alkyl is optionally substituted with one group independently selected from: —OH, C₁-C₃-alkyl-, C₁-C₃-alkyl- optionally substituted with OH, phenyl- optionally substituted with OH, and phenyl-C₁-C₃-alkyl- optionally substituted with OH;
X is O or N, provided that when X is O, R₁ is absent;
R₂ is a halogen;
R₃ is C₄-C₆-alkenyl, which is optionally substituted with one, two, or three C₁-C₃-alkyl groups, preferably 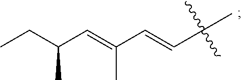 [SMILES: */C=C/C(C)=C/[C@@H](C)CC]
R₄ is —CO—C₁-C₆-alkyl, preferably CH₃CO—;
or 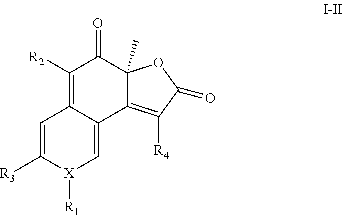
R₁ is —C₁-C₃-alkyl-COOR₇, wherein the C₁-C₃-alkyl is optionally substituted with 1 group independently selected from: —OH, —C₁-C₃-alkyl-COOR₈, and 5-10 membered heteroaryl-C₁-C₃-alkyl- containing one or two heteroatoms selected from N, O, or S optionally substituted with OH; preferably, the 5-10 membered heteroaryl is selected from: indolyl, isoindolyl, pyrazolyl, imidazolyl, pyridyl, and oxazolyl;
R₇ and R₈ are independently selected from H and C₁-C₃-alkyl-;
X is N;
R₂ is a halogen;
R₃ is C₄-C₆-alkenyl, which is optionally substituted with one, two, or three C₁-C₃-alkyl groups, preferably 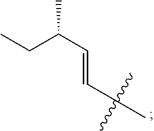 [SMILES: */C=C/[C@@H](C)CC]
R₄ is —CO—C₂-C₆-alkenyl, preferably 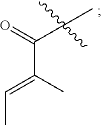 [SMILES: *C(=O)/C(C)=C/C]
or 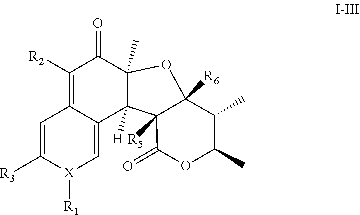
R₁ is absent
X is O;
R₂ is a halogen;
R₃ is C₄-C₆-alkenyl, which is optionally substituted with a group selected from hydroxyl and C₁-C₃-alkyl, preferably 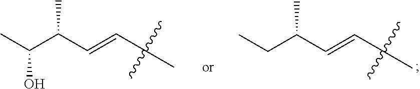
R₅ is H;
R₆ is OH;
or R₅ and R₆ are linked together to form a double bond between the two carbon atoms to which they are attached.

6. The use according to claim 1, wherein the compound of formula I is selected from the following compounds:

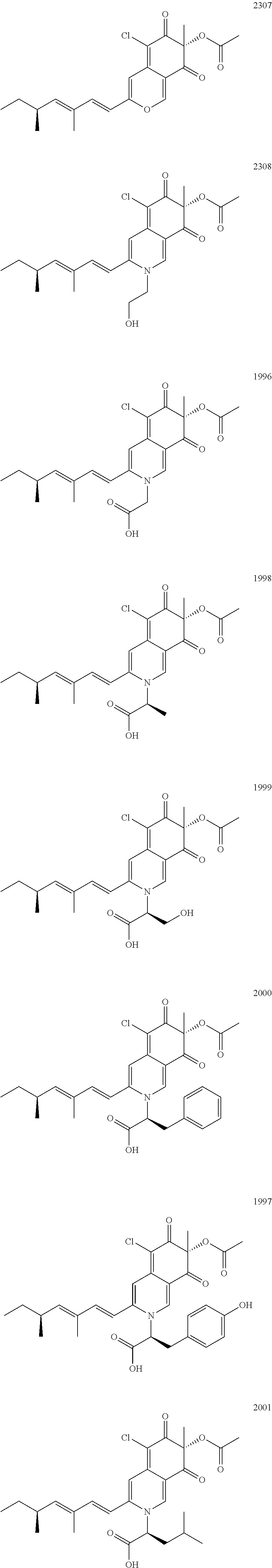

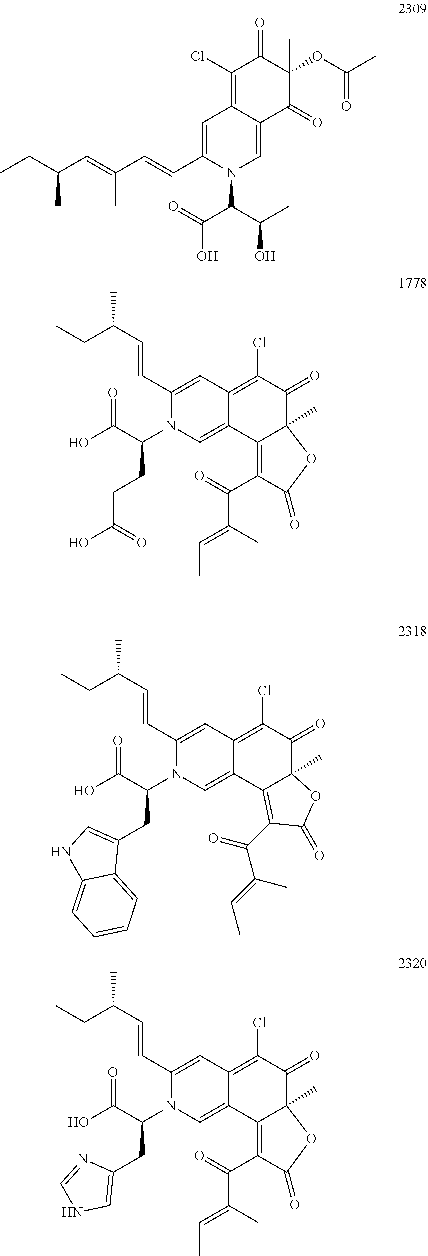

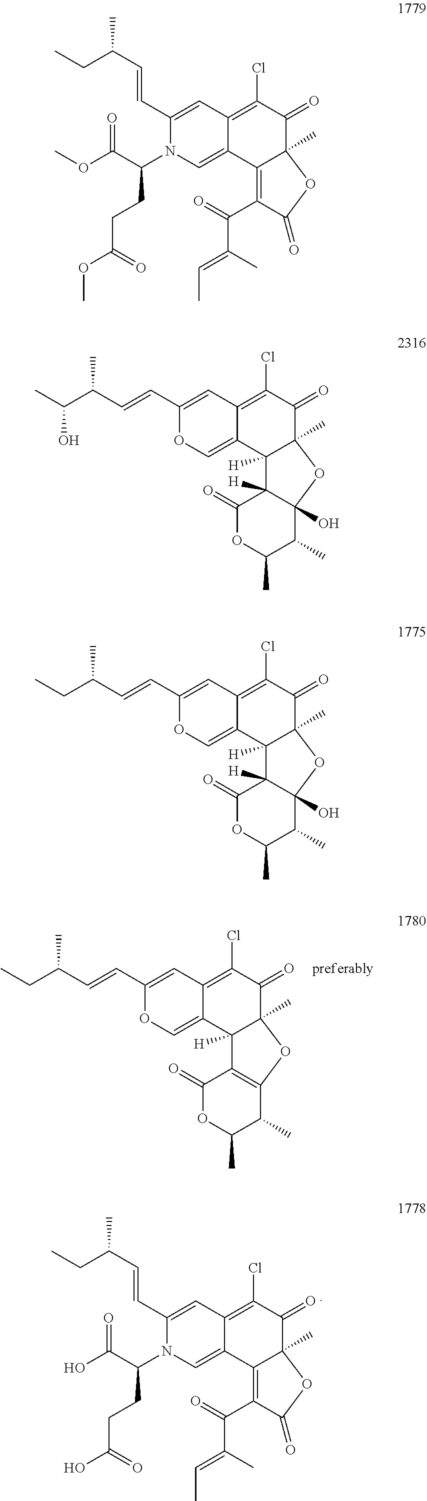

7. The use according to any one of claims 1 to 6, wherein the diseases treatable by NK cell activation include but are not limited to: tumors, autoimmune diseases, infectious diseases, and acute/chronic graft-versus-host disease, and aging;
preferably including but not limited to: tumors such as liver cancer, kidney cancer, gastric cancer, colon cancer, lung cancer, ovarian cancer, pancreatic cancer, prostate cancer, breast cancer, melanoma, thyroid cancer, glioma, multiple myeloma; leukemia such as myeloid leukemia, lymphoid leukemia, erythroid leukemia, lymphoma; autoimmune diseases such as rheumatoid arthritis, systemic lupus erythematosus, Sjögren's syndrome; viral infections such as HBV, HCV, HIV infections including hepatitis B, hepatitis C, acquired immunodeficiency syndrome (AIDS); bacterial infections: enteritis, hepatitis, pneumonia, pancreatitis, prostatitis; aging.

8. The use according to any one of claims 1 to 7, wherein the medicament is in the form of a unit dosage form, with each unit dosage form containing 1-500 mg of the compound of formula I or its pharmaceutically acceptable salts, stereoisomers.

9. Use of the compound of formula I or its pharmaceutically acceptable salts, stereoisomers according to any one of claims 1 to 6 in the preparation of a medicament to enhance the killing ability of NK cell against tumor cells.

10. A combination product comprising the compound of formula I or its pharmaceutically acceptable salts, stereoisomers according to any one of claims 1 to 6, and NK cells.

11. A compound of formula 1-1 or its pharmaceutically acceptable salts, stereoisomers 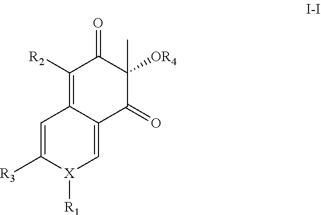
wherein
R₁ is HOC₁-C₆-alkyl-, —C₁-C₆-alkyl-COOR₇, wherein the C₁-C₆-alkyl is optionally substituted with one, two, or three groups independently selected from: —OH, C₁-C₆-alkyl-, C₁-C₆-alkyl-optionally substituted with OH, C₆-C₁₀-aryl optionally substituted with OH, C₆-C₁₀-aryl-C₁-C₆-alkyl- optionally substituted with OH, 5-10 membered heteroaryl optionally substituted with OH, and 5-10 membered heteroaryl-C₁-C₆-alkyl- optionally substituted with OH;
X is N;
R₂ is a halogen;
R₃ is selected from C₁-C₆-alkyl, C₂-C₆-alkenyl, which is optionally substituted with one, two, or three groups selected from C₁-C₃-alkyl and —OH;
R₄ is selected from —CO-C₁-C₆-alkyl and —CO—C₂-C₆-alkenyl;
R₇ is independently selected from H and C₁-C₆-alkyl.

12. The compound of formula 1-1 or its pharmaceutically acceptable salts, stereoisomers according to claim 11, wherein R₁ is HOC₁-C₃-alkyl-, —C₁-C₃-alkyl-COOR₇, wherein the C₁-C₃-alkyl is optionally substituted with one group independently selected from: —OH, C₁-C₃-alkyl-, C₁-C₃-alkyl- optionally substituted with OH, phenyl- optionally substituted with OH, phenyl-C₁-C₃-alkyl- optionally substituted with OH, 5-10 membered heteroaryl containing one or two heteroatoms selected from N, O, or S optionally substituted with OH, and 5-10 membered heteroaryl-C₁-C₃-alkyl- containing one or two heteroatoms selected from N, O, or S optionally substituted with OH; preferably, the 5-10 membered heteroaryl is selected from: indolyl, isoindolyl, pyrazolyl, imidazolyl, pyridyl, and oxazolyl;
R₃ is selected from C₄-C₆-alkyl, C₄-C₆-alkenyl, which is optionally substituted with one, two, or three C₁-C₃-alkyl or one —OH;
R₇ is independently selected from H and C₁-C₃-alkyl.

13. The compound of formula 1-1 or its pharmaceutically acceptable salts, stereoisomers according to any one of claims 11 to 12,
wherein R₁ is HOC₁-C₃-alkyl- or —C₁-C₃-alkyl-COOH, wherein the C₁-C₃-alkyl is optionally substituted with one group independently selected from: —OH, C₁-C₃-alkyl-, C₁-C₃-alkyl-optionally substituted with OH, phenyl- optionally substituted with OH, and phenyl-C₁-C₃-alkyl- optionally substituted with OH;
X is N;
R₂ is a halogen;
R₃ is C₄-C₆-alkenyl, which is optionally substituted with one, two, or three C₁-C₃-alkyl groups, preferably 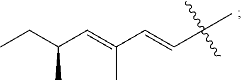 [SMILES: */C=C/C(C)=C/[C@@H](C)CC]
R₄ is —CO—C₁-C₆-alkyl, preferably CH₃CO—.

14. The compound of formula I-I or its pharmaceutically acceptable salts, stereoisomers according to claim 11, wherein R₁ is —CH₂CH₂OH, —CH₂COOH, 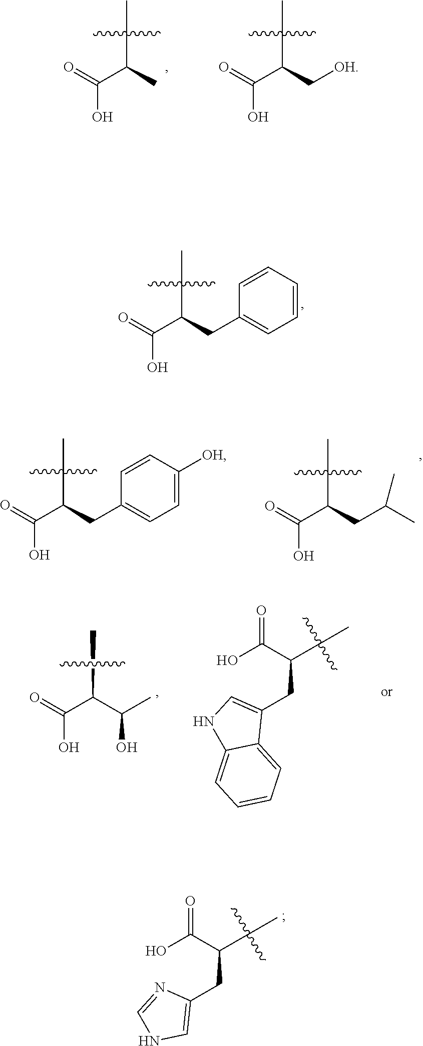
X is N;
R₂ is a halogen such as Cl;
R₃ is selected from C₄-C₆-alkenyl, which is optionally substituted with one, two, or three C₁-C₃-alkyl groups, preferably 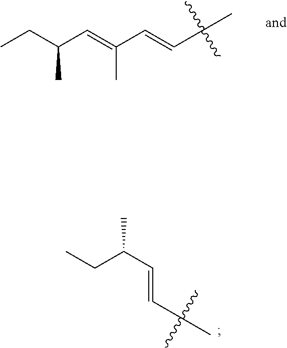
R₄ is selected from —CO—C₁-C₃-alkyl and —CO—C₂-C₄-alkenyl.

15. The compound of formula 1-1 or its pharmaceutically acceptable salts, stereoisomers according to any one of claims 11 to 14, wherein the compound of formula I-I is selected from the following compounds:

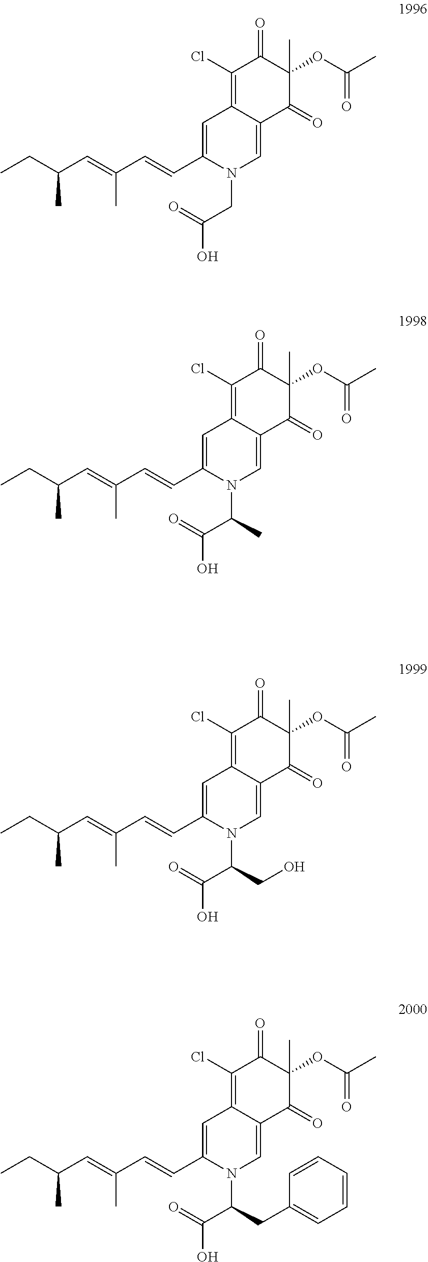

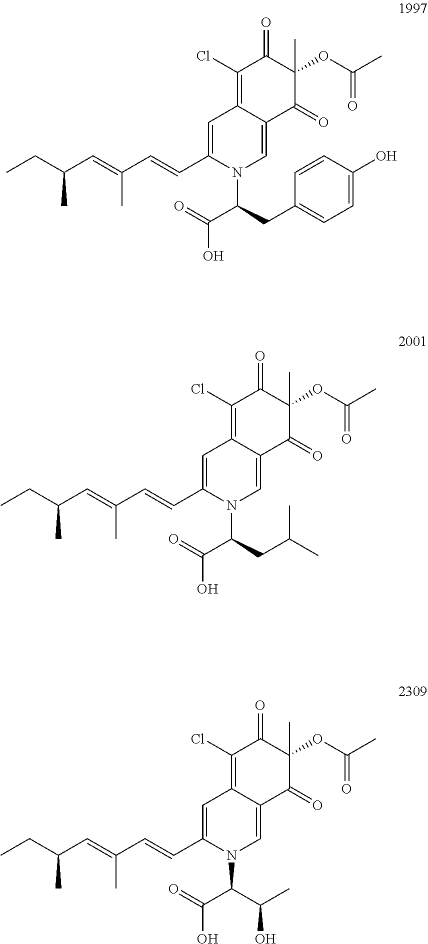

16. A pharmaceutical composition comprising the compound of formula I-I or its pharmaceutically acceptable salts, stereoisomers according to any one of claims 11 to 14, and a pharmaceutically acceptable excipient.

17. A pharmaceutical composition comprising the compound of formula I or its pharmaceutically acceptable salts, stereoisomers according to any one of claims 1 to 6, and a pharmaceutically acceptable excipient, wherein the pharmaceutical composition is in the form of a unit dosage form, with each unit dosage form containing 1-500 mg, such as 10-100 mg of the compound of formula I or its pharmaceutically acceptable salts, stereoisomers.

18. The pharmaceutical composition according to claim 16 or 17, wherein it is in one of the following forms: suspension, emulsion, tablet, capsule, granules, oral liquid, or injection.
